(19) United States
(12) Patent Application Publication
(10) Pub. No.: US 2010/0273711 A1
(43) Pub. Date: Oct. 28, 2010

(54) INDIVIDUALIZED CANCER TREATMENTS

(76) Inventors: Anil Potti, Chapel Hill, NC (US); Holly K. Dressman, Durham, NC (US); Joseph R. Nevins, Chapel Hill, NC (US)

Correspondence Address: MICHAEL BEST & FRIEDRICH LLP, 100 E WISCONSIN AVENUE, Suite 3300, MILWAUKEE, WI, 53202, US

(21) Appl. No.: 12/680,494

(22) PCT Filed: Sep. 29, 2008
(86) PCT No.: PCT/US08/78150

§ 371 Date: Jun. 22, 2010

Related U.S. Application Data
(60) Provisional application No. 60/995,910, filed on Sep. 28, 2007.

(51) Int. Cl. A61K 38/38; C12Q 1/68; A61K 31/337; A61K 31/7056; A61K 31/7048; A61K 31/517; A61K 31/7068; A61P 35/00

(52) U.S. Cl. 514/15.2; 435/6; 514/449; 514/43; 514/27; 514/266.4; 514/49

(57) ABSTRACT

Provided herein are methods for the use of gene expression profiling to determine whether an individual afflicted with cancer will respond to a therapy, and in particular to therapeutic agents such as platinum-based agents and antimetabolite agents. Methods for the treatment of individuals with the therapeutic agents are also provided. Methods of predicting the efficacy of cancer therapeutic agents such as platinum-based and antimetabolite therapeutic agents are also provided. Kits including gene chips and instructions for predicting responsiveness are also provided.

### CROSS-REFERENCE TO RELATED APPLICATIONS

This application claims priority to U.S. Provisional Application 60/995,910, filed Sep. 28, 2007, which is incorporated herein by reference in its entirety

### BACKGROUND

The National Cancer Institute has estimated that in the United States alone, one in three people will be afflicted with cancer. Moreover, approximately 50% to 60% of people with cancer will eventually die from the disease. The inability to predict responses to specific therapies is a major impediment to improving outcome for cancer patients. Because treatment of cancer typically is approached empirically, many patients with chemo-resistant disease receive multiple cycles of often toxic therapy before the lack of efficacy becomes evident. As a consequence, many patients experience significant toxicities, compromised bone marrow reserves, and reduced quality of life while receiving chemotherapy. Further, initiation of efficacious therapy is delayed.

### BRIEF SUMMARY OF THE INVENTION

Throughout this specification, reference numbering is sometimes used to refer to the full citation for the references, which can be found in the “Reference Bibliography” after the Examples section. The disclosure of all patents, patent applications, and publications cited herein are hereby incorporated by reference in their entirety for all purposes.

In one aspect, methods for predicting responsiveness of a cancer to a platinum-based chemotherapeutic agent are provided. The method includes comparing a first gene expression profile of the cancer to a platinum chemotherapy responsivity predictor set of gene expression profiles, and then predicting the responsiveness of the cancer to a platinum-based chemotherapeutic agent. The first gene expression profile and the platinum chemotherapy responsivity predictor set each comprise at least 2 genes from Table 1. Also included are methods of developing a treatment plan for an individual with cancer by administering an effective amount of a platinum-based chemotherapeutic agent to the individual with the cancer if the cancer is predicted to respond to a platinum-based chemotherapeutic agent.

In another aspect, methods of predicting responsiveness of a cancer to an antimetabolite chemotherapeutic agent are provided. These methods include comparing a first gene expression profile of the cancer to an antimetabolite chemotherapy responsivity predictor set of gene expression profiles and predicting the responsiveness of the cancer to an antimetabolite chemotherapeutic agent. The first gene expression profile and the antimetabolite chemotherapy responsivity predictor set each comprise at least 2 genes from Table 2. Also included are methods of developing a treatment plan for an individual with cancer by administering an effective amount of an antimetabolite chemotherapeutic agent to the individual with the cancer if the cancer is predicted to respond to an antimetabolite chemotherapeutic agent.

In yet another aspect, kits including a gene chip for predicting responsivity of a cancer to a platinum-based therapy comprising portions of at least 5 genes selected from Table 1 and a set of instructions for predicting responsivity of a cancer to platinum-based chemotherapeutic agents are provided.

In a further aspect, kits including a gene chip for predicting responsivity of a cancer to an antimetabolite therapeutic agent comprising portions of at least 5 genes selected from Table 2 and a set of instructions for predicting responsivity of a cancer to antimetabolite therapeutic agents.

In a still further aspect, computer readable mediums including gene expression profiles and corresponding responsivity information for platinum-based chemotherapeutic agents or antimetabolite chemotherapeutic agents comprising at least 5 genes from any of Tables 1 or 2 are provided.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A and 1B depict a gene expression pattern associated with platinum response and a gene expression pattern associated with pemetrexed response, respectively. FIG. 1A, left panel, shows the expression plot for genes predicting cisplatin resistance or sensitivity, where blue represents low expression and red represents high expression. Each column represents one cell line and each row represents one gene. The right panel shows results from a leave-one-out cross validation of the training set (blue=Incomplete Responders, red=Responders). FIG. 1B, left panel, shows the expression plot for genes predicting pemetrexed resistance or sensitivity, where blue represents low expression and red represents high expression. Each column represents one cell line and each row represents one gene. The right panel shows results from a leave-one-out cross validation of the training set (blue=Incomplete Responders, red=Responders).

FIGS. 2A and 2B depict in vitro validation of the cisplatin and pemetrexed predictors for tumor cell lines. FIG. 2A shows the IC50 of cisplatin (y axis) plotted against predicted sensitivity to cisplatin (x-axis) in cell lines. The graph demonstrates that the model predicts sensitivity to cisplatin (p<0.001 (left panel: ovarian cancer lines) and p=0.03 (right panel: lung cancer lines)). FIG. 2B shows the IC50 of pemetrexed (y-axis) plotted against the predicted probability of sensitivity to pemetrexed (x-axis). The graph demonstrates that the model predicts sensitivity to pemetrexed in NSCLC lines (p=0.0006).

FIG. 3 depicts in vivo validation of the cisplatin sensitivity predictor set for patient outcomes. Patients were classified as responders and non-responders as defined in Example 1. The top panel represents the predicted probability of cisplatin sensitivity and the bottom panel represents the probability of sensitivity to cisplatin (p<0.01).

FIGS. 4A and 4B depict the negative correlation between cisplatin sensitivity and pemetrexed sensitivity in lung cancer tumors (A) and cell lines (B). The top row represents the probability of cisplatin resistance (blue=sensitive, red=resistant) and the bottom row represents the corresponding probability of pemetrexed resistance for each sample. The right panels show plots of the predicted resistance to cisplatin versus the predicted resistance to pemetrexed and the negative correlation between said values (p=0.004) and cell lines (p=0.01).

FIGS. 5A and 5B illustrate that the sequence of chemotherapy may be critical in optimizing patient responses. FIG. 5A demonstrates that there is a negative correlation between the IC50s of various lung cancer cell lines to cisplatin and pemetrexed. FIG. 5B illustrates the experimental sequence where the pemetrexed-sensitive NSCLC cell line (H2030) develops resistance to pemetrexed after exposure to a taxane for 4 days.

FIG. 6 depicts a decision-making strategy for treating patients with advanced NSCLC utilizing a platinum-based chemotherapy sensitivity predictor.

### BRIEF DESCRIPTION OF THE TABLES

Table 1 lists the 45 genes that contribute the most weight in the prediction and that appeared most often within the models for platinum-based responsivity predictor set.

Table 2 lists the 85 genes that contribute the most weight in the prediction and that appeared most often within the models for antimetabolite responsivity predictor set.

Table 3 lists the cell lines used to generate the platinum-based chemotherapy predictor set and an indication of whether the cell line was sensitive or resistant to treatment with cisplatin.

Table 4 lists the cell lines used to generate the antimetabolite chemotherapy predictor set and an indication of whether the cell line was sensitive or resistant to treatment with pemetrexed.

### DETAILED DESCRIPTION OF THE INVENTION

Individuals with ovarian cancer frequently progress to an advanced stage before any symptoms appear. The standard treatment for advanced stage (e.g., Stage III/IV) ovarian cancer is to combine cytosurgery (e.g., “debulking” the individual of the tumor) with a platinum-based treatment. In some cases, carboplatin or cisplatin is administered. Other non-limiting alternatives to carboplatin and cisplatin are oxaliplatin and nedaplatin. Taxane is sometimes administered with the carboplatin or cisplatin.

Platinum based treatment is not effective for all patients. Thus, physicians must consider alternative treatments to combat cancer. Alternative therapeutic agents include, but are not limited to, denopterin, edatrexate, methotrexate, nolatrexed, pemetrexed, piritrexim, pteropterin, raltitrexed, trimetrexate, cladribine, clofarabine, fludarabine, 6-mercaptopurine, nelarabine, thiamiprine, thioguanine, tiazofurin, ancitabine, azacitidine, 6-azauridine, capecitabine, carmofur, cytarabine, decitabine, doxifluridine, enocitabine, floxuridine, fluorouracil, gemcitabine, tegafur, troxacitabine, pentostatin, hydroxyurea, cytosine arabinoside, docetaxel, paclitaxel, abraxane, topotecan, adriamycin, etoposide, fluorouracil (5-FU), and cyclophosphamide. In one embodiment, the agent may be selected from alkylating agents (e.g., nitrogen mustards), antimetabolites (e.g., pyrimidine analogs), radioactive isotopes (e.g., phosphorous and iodine), miscellaneous agents (e.g., substituted ureas) and natural products (e.g., vinca alkyloids and antibiotics). In another embodiment, the therapeutic agent may be selected from the group consisting of allopurinol sodium, dolasetron mesylate, pamidronate disodium, etidronate, fluconazole, epoetin alfa, levamisole HeL, amifostine, granisetron HCL, leucovorin calcium, sargramostim, dronabinol, mesna, filgrastim, pilocarpine HCl, octreotide acetate, dexrazoxane, ondansetron HCL, ondanselron, busulfan, carboplatin, cisplatin, thiotepa, melphalan HCl, melphalan, cyclophosphamide, ifosfamide, chlorambucil, mechlorethamine HCL, carmustine, lomustine, polifeprosan 20 with carmustine implant, streptozocin, doxorubicin HCL, bleomycin sulfate, daunirubicin HCL, dactinomycin, daunorucbicin citrate, idarubicin HCL, pllmycin, mitomycin, pentostatin, mitoxantrone, valrubicin, cytarabine, tludarabine phosphate, floxuridine, cladribine, methotrexate, mercaptipurine, thioguanine, capecitabine, methyltestosterone, nilutamide, testolactone, bicalutamide, flutamide, anastrozole, toremifene citrate, estramustine phosphate sodium, ethinyl estradiol, estradiol, esterified estrogens, conjugated estrogens, leuprolide acetate, goserelin acetate, medroxyprogesterone acetate, megestrol acetate, levamisole HCL, aldesleukin, irinotecan HCL, dacarbazine, asparaginase, etoposide phosphate, gemcitabine HCL, altretamine, topotecan HCL, hydroxyurea, interferon alpha-2b, mitotane, procarbazine HCL, vinorelbine tartrate, E. coli 1-asparaginase, Erwinia L-asparaginase, vincristine sulfate, denileukin diftitox, aldesleukin, rituximab, interferon alpha-1a, paclitaxel, abraxane, docetaxel, BCG live (intravesical), vinblastine sulfate, etoposide, tretinoin, teniposide, porfuner sodium, tluorouracil, betamethasone sodium phosphate and betamethasone acetate, letrozole, etoposide citrororum factor, folinic acid, calcium leucouorin, 5-fluorouricil, adriamycin, c}toxan, and diamino-dichloro-platinum.

The difficulty with administering one or more alternative therapeutic agent is that not all individuals with cancer will respond favorably to the alternative therapeutic agent selected by the physician. Frequently, the administration of one or more alternative therapeutic agent results in the individual becoming even more ill from the toxicity of the agent and the cancer still persists. Due to the cytotoxic nature of the many chemotherapeutic agents, the individual is physically weakened and his/her immunologically compromised system cannot generally tolerate multiple rounds of “trial and error” type of therapy. Hence a treatment plan that is personalized for the individual is highly desirable.

As described in the Examples, the inventors applied genomic methodologies to identify gene expression patterns within primary tumors that predict response to primary platinum-based chemotherapy. Gene expression patterns were also identified that predict response to antimetabolite therapies. The invention also provides integrating gene expression profiles that predict platinum-response and antimetabolite response as a strategy for developing personalized treatment plans for individual patients.

About 80% of lung cancers are classified as non small cell lung cancer (NSCLC) and are divided into three main groups, squamous cell lung carcinoma, adenocarcinoma, and large cell lung carcinoma. Each type has a similar prognosis and is treated with similar therapies including chemotherapy, radiation therapy, and surgery. In advanced NSCLC, third generation regimens consisting of a platinum analog in combination with a second agent increases overall response and survival when compared to older regimens.^{1,2,3} However, overall response is still only 20-30%,³ suggesting that a majority of the patients do not respond to a platinum analog. Subsequently, those patients who fail platinum-based therapy typically receive pemetrexed, docetaxel, or targeted therapies as second line treatment, with response rates of around 7-10%.^{4,5,6} As discussed above, patients cannot tolerate multiple rounds of trial and error therapy. Individualized treatment plans are needed.

“Platinum-based therapy” and “platinum-based chemotherapy” are used interchangeably herein and refer to agents or compounds that are associated with platinum. These agents include, but are not limited to cisplatin, carboplatin, oxalipatin and nedaplatin.

“Antimetabolite therapy” and “antimetabolite chemotherapy” are used interchangeably herein and refer to agents or compounds that block nucleotide production and interfere with DNA replication and/or RNA synthesis. Antimetabolite agents include, but are not limited to, denopterin, edatrexate, methotrexate, nolatrexed, pemetrexed, piritrexim, pteropterin, raltitrexed, trimetrexate, cladribine, clofarabine, fludarabine, 6-mercaptopurine, nelarabine, thiamiprine, thioguanine, tiazofurin, ancitabine, azacitidine, 6-azauridine, capecitabine, cannofur, cytarabine, decitabine, doxifluridine, enocitabine, floxuridine, fluorouracil, gemcitabine, tegafur, troxacitabine, pentostatin, hydroxyurea, and cytosine arabinoside.

A “complete response” (CR) to treatment of cancer is defined as a complete disappearance of all measurable and assessable disease. In ovarian cancer a complete response includes, in the absence of measurable lesions, a normalization of the CA-125 level following adjuvant therapy. An individual who exhibits a complete response is known as a “complete responder.”

An “incomplete response” (IR) includes those who exhibited a “partial response” (PR), had “stable disease” (SD), or demonstrated “progressive disease” (PD) during primary therapy.

A “partial response” refers to a response that displays 50% or greater reduction in bi-dimensional size (area) of the lesion for at least 4 weeks or, in ovarian cancer, a drop in the CA-125 level by at least 50% for at least 4 weeks.

“Progressive disease” refers to response that is a 50% or greater increase in the product from any lesion documented within 8 weeks of initiation of therapy, the appearance of any new lesion within 8 weeks of initiation of therapy, or in the case of ovarian cancer, any increase in the CA-125 from baseline at initiation of therapy.

“Stable disease” was defined as disease not meeting any of the above criteria.

“Effective amount” refers to an amount of a chemotherapeutic agent that is sufficient to exert a prophylactic or therapeutic effect in the subject, i.e., that amount which will stop or reduce the growth of the cancer or cause the cancer to become smaller in size compared to the cancer before treatment or compared to a suitable control. In most cases, an effective amount will be known or available to those skilled in the art. The result of administering an effective amount of a chemotherapeutic agent may lead to effective treatment of the patient. It is desirable for an effective amount to be an amount sufficient to exert cytotoxic effects on cancerous cells.

“Predicting” and “prediction” as used herein includes, but is not limited to, generating a statistically based indication of whether a particular chemotherapeutic agent will be effective to treat the cancer. This does not mean that the event will happen with 100% certainty.

As used herein, “individual” and “subject” are interchangeable. A “patient” refers to an “individual” who is under the care of a treating physician.

The present invention may be practiced using techniques known to those skilled in the art. Such techniques are available in the literature or in scientific treatises, such as, Molecular Cloning: A Laboratory Manual, second edition (Sambrook et al., 1989) and Molecular Cloning: A Laboratory Manual, third edition (Sambrook and Russel, 2001), (jointly referred to herein as “Sambrook); Current Protocols in Molecular Biology (F. M. Ausubel et al., eds., 1987, including supplements); PCR: The Polymerase Chain Reaction, (Mullis et al., eds., 1994); Harlow and Lane (1988) Antibodies, A Laboratory Manual, Cold Spring Harbor Publications, New York; Harlow and Lane (1999) Using Antibodies: A Laboratory Manual Cold Spring Harbor Laboratory Press, Cold Spring Harbor, N.Y. (jointly referred to herein as “Harlow and Lane”), Beaucage et al. eds., Current Protocols in Nucleic Acid Chemistry John Wiley & Sons; Inc., New York, 2000) and Casarett and Doull's Toxicology The Basic Science of Poisons, C. Klaassen, ed., 6th edition (2001).

### Methods for Predicting Responsiveness to Chemotherapy

Methods of predicting responsiveness of a cancer to a chemotherapeutic agent are provided herein. Specifically, the methods rely on comparing a gene expression profile of the cancer to a chemotherapy responsivity predictor set. See Table 1 and 2 for cisplatin and pemetrexed responsivity predictor sets, respectively. The chemotherapy responsivity predictor set is expected to be distinct for each class of chemotherapeutic agents and may be somewhat altered between chemotherapeutic agents within the same class. A class of chemotherapeutic agents is a set of chemotherapeutic agents which are similar in some way. For example, the agents may be known to act through a similar mechanism, have similar targets or similar structures.

The chemotherapy predictor set is, or may be derived from, a set of gene expression profiles obtained from samples (cell lines, tumor samples, etc.) with known sensitivity or resistance to the chemotherapeutic agent. The comparison of the expression of a specific set of genes in the cancer to the same set of genes in samples known to be sensitive or resistant to the chemotherapeutic agent allows prediction of the responsiveness of the cancer to the chemotherapeutic agent. The prediction may indicate that the cancer will respond completely to the chemotherapeutic agent, or it may predict that the cancer will be only partially responsive or non-responsive to the chemotherapeutic agent. The cell lines used to generate the chemotherapy responsivity predictor sets and an indication of the cell lines' sensitivity or resistance to cisplatin or pemetrexed are provided in Tables 3 and 4, respectively.

The methods described herein provide an indication of whether the cancer in the patient is likely to be responsive to a particular chemotherapeutic prior to beginning treatment that is more accurate than predictions using population-based approaches from clinical studies. The methods allow identification of chemotherapeutics estimated to be useful in combating a particular cancer in an individual patient, resulting in a more cost-effective, targeted therapy for the cancer patient and avoiding side effects from non-efficacious chemotherapeutic agents.

Tables 1 and 2 provide the relative “weights” of each of the individual genes that make up the responsivity predictor set. The weights demonstrate that some genes are more strongly indicative of sensitivity or resistance of a cancer to a particular therapeutic agent. Predictions based on the complete set of genes are expected to provide the most accurate predictions regarding the efficacy of treating the cancer with a particular therapeutic agent. Those of skill in the art will understand based on the weights of each gene in the responsivity predictor set that some genes are more predictive of outcome than others and thus that the entire responsivity predictor set need not be used to develop a prediction.

Once an individual's cancer is predicted to be responsive to a particular chemoptherapy, then a treatment plan can be developed incorporating the chemotherapeutic agent and an effective amount of the chemotherapeutic agent(s) may be administered to the individual with the cancer. Those of skill in the art will appreciate that the methods do not guarantee that the individuals will be responsive to the chemotherapeutic agent, but the methods will increase the probability that the selected treatment will be effective to treat the cancer.

Treatment or treating a cancer includes, but is not limited to, reduction in cancer growth or tumor burden, enhancement of an anti-cancer immune response, induction of apoptosis of cancer cells, inhibition of angiogenesis, enhancement of cancer cell apoptosis, and inhibition of metastases. Administration of an effective amount of a chemotherapeutic agent to a subject may be carried out by any means known in the art including, but not limited to intraperitoneal, intravenous, intramuscular, subcutaneous, transcutaneous, oral, nasopharyngeal or transmucosal absorption. The specific amount or dosage administered in any given case will be adjusted in accordance with the specific cancer being treated, the condition, including the age and weight, of the subject, and other relevant medical factors known to those of skill in the art.

In one embodiment, the individual has cancer. Cancers include but are not limited to any cancer treatable with a platinum-based or antimetabolite therapy. Cancers include, but are not limited to, ovarian cancer, lung cancer, and breast cancer. In another embodiment, the individual has advanced stage cancer (e.g., Stage III/IV ovarian cancer). In other embodiments, the individual has early stage cancer whereby cellular samples from the early stage ovary cancer are obtained from the individual. For the individuals with advanced cancer, one form of primary treatment practiced by treating physicians is to surgically remove as much of the tumor as possible, a practice sometime known as “debulking ” The sample of the cancer used to obtain the first gene expression profile may be directly from a tumor that was surgically removed. Alternatively, the sample of the cancer could be from cells obtained in a biopsy or other tumor sample. A sample from ascites surrounding the tumor may also be used.

The sample is then analyzed to obtain a first gene expression profile. This can be achieved by any means available to those of skill in the art. One method that can be used is to isolate RNA (e.g., total RNA) from the cellular sample and use a publicly or commercially available micro array system to analyze the gene expression profile from the cellular sample. One microarray that may be used is Affymetrix Human U133A chip. One of skill in the art follows the standard directions that come with a commercially available microarray. Other types of microarrays may be used, for example, microarrays using RT-PCR for measurement. Other sources of microarrays include, but are not limited to, Stratagene (e.g., Universal Human Microarray), Genomic Health (e.g., Oncotype DX chip), Clontech (e.g., Atlas™ Glass Microarrays), and other types of Affymetrix microarrays. In one embodiment, the microarray comes from an educational institution or from a collaborative effort whereby scientists have made their own microarrays. In other embodiments, customized microarrays, which include the particular set of genes that are particularly suitable for prediction, can be used.

Once a first gene expression profile has been obtained from the sample, it is compared with chemotherapy responsivity predictor set of gene expression profiles. Two such chemotherapy responsivity predictor sets are disclosed herein, a platinum-based chemotherapy responsivity predictor set and an antimetabolite chemotherapy responsivity predictor set.

### Chemotherapy Responsivity Predictor Set of Gene Expression Profiles

A pemetrexed chemotherapy responsitivity predictor set was created by a method described in detail in the Examples and similar to that detailed in Potti et al. (Genomic signatures to guide the use of chemotherapeutics. Nature Medicine 12(11): 1294-1300, 2006, incorporated herein by reference). The [−log10(M)] GI50/IC50 and LC50 (50% cytotoxic dose) data on the NCI-60 cell line panel for pemetrexed was used to populate a matrix with MATLAB software with the relevant expression data for each individual cell line. When multiple entries for a drug screen existed (by NCS number), the entry with the largest number of replicates was included. To develop in vitro gene expression based predictor of pemetrexed sensitivity from the pharmacologic data used in the NCI-60 drug screen studies, we chose cell lines within the NCI-60 panel that would represent the extremes of sensitivity (See Table 4). Relevant expression data (updated data available on the Affymetrix U95A2 GeneChip) for the selected NCI-60 cell lines were then used in a supervised analysis using Bayesian regression methodologies, as described previously (Pittman J, Huang E, Nevins J, et al: Bayesian analysis of binary prediction tree models for retrospectively sampled outcomes. Biostatistics 5(4):587-601, 2004), to develop a probit model predictive of sensitivity to pemetrexed.

The collection of data in the NCI-60 data occasionally does not represent a significant diversity in resistant and sensitive cell lines to any given drug. Thus, if a drug screening experiment did not result in widely variable GI50/IC50 and/or LC50 data, the generation of a genomic predictor is not possible using our methods, as was the case for cisplatin. Thus, data published by Gyorffy et al. (Gyorffy B, et al: Gene expression profiling of 30 cancer cell lines predicts resistance towards 11 anticancer drugs at clinically achieved concentrations. Int J Cancer 118(7): 1699-1712, 2005, which is incorporated by reference herein in its entirety) was used. Gyorffy determined definitive resistance and sensitivity to cisplatin in 30 cancer cell lines. All array data are available on the supplemental website (data.cgt.duke.edu/JCO.php). The cell lines and the sensitivity to cisplatin are indicated in Table 3.

Thus, one of skill in art may use the chemotherapy responsitivity predictor set as detailed in Example 1 or in Example 2 to predict whether the first gene expression profile, obtained from the individual or patient with cancer will be responsive to a platinum-based therapy or an antimetabolite therapy. If the individual is a complete responder to a platinum-based chemotherapeutic agent, then a platinum-based therapeutic agent will be administered in an effective amount, as determined by the treating physician. Likewise, if the individual is a complete responder to an antimetabolite chemotherapeutic agent, then an antimetabolite therapeutic agent will be administered in an effective amount, as determined by the treating physician. If the complete responder stops being a complete responder, as sometimes happens, then the first gene expression profile may be further analyzed for responsivity to an alternative agent to determine which alternative agent should be administered to most effectively combat the cancer while minimizing the toxic side effects to the individual. If the individual is an incomplete responder, then the individual's gene expression profile can be further analyzed for responsivity to an alternative agent to determine which agent should be administered.

The use of the chemotherapy responsitivity predictor set in its entirety is contemplated; however, it is also possible to use subsets of the predictor set. For example, a subset of at least 2, 5, 10, 15, 20, 25, 30, 35 or 40 or more genes from Tables 1 or 2 can be used for predictive purposes. For example, 40, 45, 50, 55, 60, 65, 70, 75 or 80 genes from Table 2 could be used in an antimetabolite chemotherapy responsivity predictor set.

Thus, in this manner, an individual can be evaluated for responsiveness to either a platinum-based or an antimetabolite chemotherapeutic agent. In certain embodiments, the methods of the application are performed outside of the human body. In addition, an individual can be assessed to determine if they will be refractory to platinum-based therapy or antimetabolite therapy such that additional alternative therapeutic intervention can be started.

For the individuals that appear to be incomplete responders to platinum-based therapy or for those individuals who have ceased being complete responders, an important step in the treatment is to determine what other alternative cancer therapies might be given to the individual to best combat the cancer while minimizing the toxicity of these additional agents.

In one aspect, alternative chemotherapeutic agents may be used. These alternative agents include, but are not limited to, denopterin, edatrexate, methotrexate, nolatrexed, pemetrexed, piritrexim, pteropterin, raltitrexed, trimetrexate, cladribine, clofarabine, fludarabine, 6-mercaptopurine, nelarabine, thiamiprinc, thioguanine, tiazofurin, ancitabine, azacitidine, 6-azauridine, capecitabine, carmofur, cytarabine, decitabine, doxifluridine, enocitabine, floxuridine, fluorouracil, gemcitabine, tegafur, troxacitabine, pentostatin, hydroxyurea, cytosine arabinoside, topotecan, adriamycin, doxorubicin, cytoxan, cyclophosphamide, gemcitabine, etoposide, ifosfamide, paclitaxel, abraxane, docetaxel, and taxol.

In another aspect, the first gene expression profile from the individual with cancer is analyzed and compared to gene expression profiles (or signatures) that are reflective of deregulation of various oncogenic signal transduction pathways. In one embodiment, the alternative cancer therapeutic agent is directed to a target that is implicated in oncogenic signal transduction deregulation. Such targets include, but are not limited to, Src, myc, beta-catenin and E2F3 pathways. Thus, in one aspect, the invention contemplates using an inhibitor that is directed to one of these targets as an additional therapy for cancer. One of skill in the art will be able to determine the dosages for each specific chemotherapeutic agent.

In one aspect, the alternative agent is an antimetabolite. Antimetabolites are small molecules that interfere with the enzymatic synthesis of crucial organic molecules such as nucleotides. Examples of antimetabolites include, but are not limited to, denopterin, edatrexate, methotrexate, nolatrexed, pemetrexed, piritrexim, pteropterin, raltitrexed, trimetrexate, cladribine, clofarabine, fludarabine, 6-mercaptopurine, nelarabine, thiamiprine, thioguanine, tiazofurin, ancitabine, azacitidine, 6-azauridine, capecitabine, cannofur, cytarabine, decitabine, doxifluridine, enocitabine, floxuridine, fluorouracil, gemcitabine, tegafur, troxacitabine, pentostatin, hydroxyurea, and cytosine arabinoside.

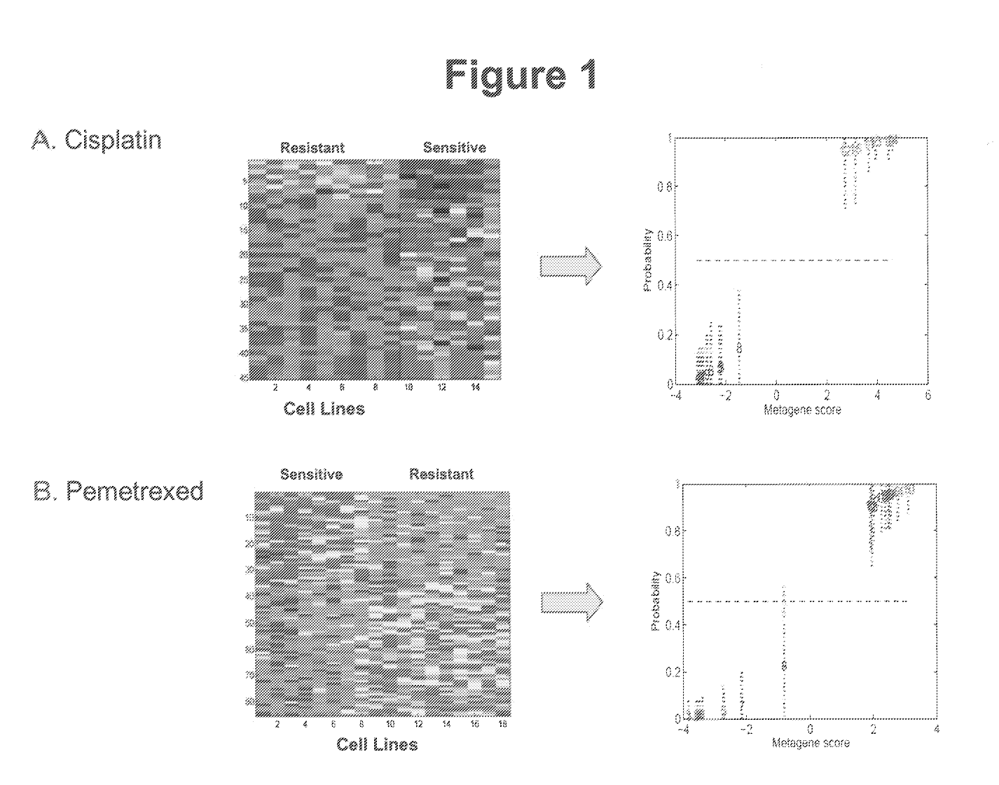

As shown in Example 1, the teachings herein provide a gene expression model that predicts response to platinum-based therapy. The gene expression model was developed by using Bayesian binary regression analysis to identify genes highly correlated with drug sensitivity. The developed model consisting of 45 genes based on cisplatin sensitivity (FIG. 1a) and was validated in a leave-one-out cross validation. The cisplatin sensitivity predictor includes DNA repair genes such as ERCC 1 and ERCC4 among others that had altered expression in the list of cisplatin sensitivity predictor genes. (Table 1).

As shown in Example 2, the predictor set to determine responsitivity to pemetrexed is shown in Table 2. As with the platinum-based predictor set, in certain embodiments, not all of the genes in the pemetrexed predictor must be used. A subset comprising at least 5, 10, or 15 genes may be used as a predictor set to predict responsivity to pemetrexed.

Method of Treating Individuals with Cancer

The methods described herein also include treating an individual afflicted with cancer. This method involves administering an effective amount of a platinum-based therapy to those individuals predicted to be responsive to such therapy. In the alternative, an effective amount of an antimetabolite therapy may be administered to individuals predicted to be responsive to that therapy. In the instance where the individual is predicted to be a non-responder, a physician may decide to administer alternative therapeutic agents alone. In many instances, the treatment will comprise a combination of a platinum-based therapy or an antimetabolite therapy and an alternative agent. In one embodiment, the treatment will comprise a combination of a platinum-based therapy and an inhibitor of a signal transduction pathway that is deregulated in the individual with cancer.

The methods described herein include, but are not limited to, treating individuals afflicted with NSCLC, breast cancer and ovarian cancer. In one aspect, platinum-based therapy or an antimetabolite therapy are administered in an effective amount by themselves (e.g., for complete responders). In another embodiment, the therapeutic agent is administered with an alternative chemotherapeutic in an effective amount concurrently. In another embodiment, the two therapeutic agents are administered in an effective amount in a sequential manner. In yet another embodiment, the alternative therapeutic agent is administered in an effective amount by itself In yet another embodiment, the alternative therapeutic agent is administered in an effective amount first and then followed concurrently or step-wise by a platinum-based therapeutic agent or an antimetabolite therapeutic agent.

Methods of Predicting/Estimating the Efficacy of a Therapeutic Agent in Treating an Individual Afflicted with Cancer

One aspect of the invention provides a method for predicting, estimating, aiding in the prediction of, or aiding in the estimation of, the efficacy of a therapeutic agent in treating a subject afflicted with cancer. In certain embodiments, the methods of the application are performed outside of the human body.

One method comprises (a) determining the expression level of multiple genes in a tumor biopsy sample from the subject; (b) defining the value of one or more metagenes from the expression levels of step (a), wherein each metagene is defined by extracting a single dominant value using singular value decomposition (SVD) from a chemotherapy responsivity predictor set; and (c) averaging the predictions of one or more statistical tree models applied to the values of the metagenes, wherein each model includes one or more nodes, each node representing a metagene, each node including a statistical predictive probability of tumor sensitivity to the therapeutic agent, thereby estimating the efficacy of a therapeutic agent in a subject afflicted with cancer. Another method comprises (a) determining the expression level of multiple genes in a tumor biopsy sample from the subject; (b) defining the value of one or more metagenes from the expression levels of step (a), wherein each metagene is defined by extracting a single dominant value using singular value decomposition (SVD) from a chemotherapy responsivity predictor set; and (c) averaging the predictions of one or more binary regression models applied to the values of the metagenes, wherein each model includes a statistical predictive probability of tumor sensitivity to the therapeutic agent, thereby estimating the efficacy of a therapeutic agent in a subject afflicted with cancer.

In one embodiment, the predictive methods of the invention predict the efficacy of a therapeutic agent in treating a subject afflicted with cancer with at least 70% accuracy. In another embodiment, the methods predict the efficacy of a therapeutic agent in treating a subject afflicted with cancer with at least 80% accuracy. In another embodiment, the methods predict the efficacy of a therapeutic agent in treating a subject afflicted with cancer with at least 85% accuracy. In another embodiment, the methods predict the efficacy of a therapeutic agent in treating a subject afflicted with cancer with at least 90% accuracy. In another embodiment, the methods predict the efficacy of a therapeutic agent in treating a subject afflicted with cancer with at least 70%, 75%, 80%, 85%, 90% or 95% accuracy when tested against a validation sample. In another embodiment, the methods predict the efficacy of a therapeutic agent in treating a subject afflicted with cancer with at least 70%, 75%, 80%, 85%, 90% or 95% accuracy when tested against a set of training samples. In another embodiment, the methods predict the efficacy of a therapeutic agent in treating a subject afflicted with cancer with at least 70%, 75%, 80%, 85%, 90% or 95% accuracy when tested on human primary tumors ex vivo or in vivo. Accuracy is the ability of the methods to predict whether a cancer is sensitive or resistant to the chemotherapeutic agent.

The predictive methods predict the efficacy of a therapeutic agent to treat a subject with cancer with at least 70%, 75%, 80%, 85%, 90%, 95%, 97%, 98%, 99% or 100% sensitivity for a particular chemotherapeutic agent. In another embodiment, the methods predict the efficacy of a therapeutic agent in treating a subject afflicted with cancer with at least 70%, 75%, 80%, 85%, 90%, 95%, 97%, 98%, 99% or 100% sensitivity when tested against a validation sample. In another embodiment, the methods predict the efficacy of a therapeutic agent in treating a subject afflicted with cancer with at least 70%, 75%, 80%, 85%, 90%, 95%, 97%, 98%, 99% or 100% sensitivity when tested against a set of training samples. In another embodiment, the methods predict the efficacy of a therapeutic agent in treating a subject afflicted with cancer with at least 70%, 75%, 80%, 85%, 90%, 95%, 97%, 98%, 99% or 100% sensitivity when tested on human primary tumors ex vivo or in vivo. Sensitivity measure the ability of the methods to predict all cancers that will be sensitive to the chemotherapeutic agent.

### (A) Sample of the Cancer

In one embodiment, the predictive methods of the invention comprise determining the expression level of genes in a tumor sample from the subject. In certain embodiments, the tumor is a breast tumor, an ovarian tumor, or a lung tumor. In one embodiment, the tumor is not a breast tumor. In one embodiment, the tumor is not an ovarian tumor. In one embodiment, the tumor is not a lung tumor. In one embodiment of the methods described herein, the methods comprise the step of surgically removing a tumor sample from the subject, obtaining a tumor sample from the subject, or providing a tumor sample from the subject.

Alternatively, the sample may be derived from cells from the cancer, or cancerous cells. In another embodiment, the cells may be from ascites surrounding the tumor. The sample may contain nucleic acids from the cancer. Any method may be used to remove the sample from the patient.

In one embodiment, at least 40%, 50%, 60%, 70%, 80% or 90% of the cells in the sample are cancer cells. In preferred embodiments, samples having greater than 50% cancer cell content are used. In one embodiment, the sample is a live tumor sample. In another embodiment, the sample is a frozen sample. In one embodiment, the sample is one that was frozen within less than 5, 4, 3, 2, 1, 0.75, 0.5, 0.25, 0.1, 0.05 or less hours after extraction from the patient. Frozen samples include those stored in liquid nitrogen or at a temperature of about −80° C. or below.

### (B) Gene Expression

The expression of the genes may be determined using any method known in the art for assaying gene expression. Gene expression may be determined by measuring mRNA or protein levels for the genes. In one embodiment, an mRNA transcript of a gene may be detected for determining the expression level of the gene. Based on the sequence information provided by the GenBank™ database entries, the genes can be detected and expression levels measured using techniques well known to one of ordinary skill in the art, including but not limited to rtPCR, Northern blot analysis and microarray analysis. For example, sequences within the sequence database entries corresponding to polynucleotides of the genes can be used to construct probes for detecting mRNAs by, e.g., Northern blot hybridization analyses. The hybridization of the probe to a gene transcript in a subject biological sample can be also carried out on a DNA array. The use of an array is suitable for detecting the expression level of a plurality of the genes. As another example, the sequences can be used to construct primers for specifically amplifying the polynucleotides in, e.g., amplification-based detection methods such as reverse-transcription based polymerase chain reaction (RT-PCR). As another example, mRNA levels can be assayed by quantitative RT-PCR. Furthermore, the expression level of the genes can be analyzed based on the biological activity or quantity of proteins encoded by the genes. Methods for determining the quantity of the protein include immunoassay methods such as Western blot analysis.

In one exemplary embodiment, about 1-50 mg of cancer tissue is added to a chilled tissue pulverizer, such as to a BioPulverizer H tube (Bio101 Systems, Carlsbad, Calif.). Lysis buffer, such as from the Qiagen RNeasy Mini kit, is added to the tissue and homogenized. Devices such as a Mini-Beadbeater (Biospec Products, Bartlesville, Okla.) may be used. Tubes may be spun briefly as needed to pellet the garnet mixture and reduce foam. The resulting lysate may be passed through syringes, such as a 21 gauge needle, to shear DNA. Total RNA may be extracted using commercially available kits, such as the Qiagen RNeasy Mini kit. The samples may be prepared and arrayed using Affymetrix U133 plus 2.0 GeneChips or Affymetrix U133A GeneChips. Any suitable gene chip may be used.

In one exemplary embodiment, total RNA was extracted using the Qiashredder and Qiagen RNeasy Mini kit and the quality of RNA was checked by an Agilent 2100 Bioanalyzer. The targets for Affymetrix DNA microarray analysis were prepared according to the manufacturer's instructions. Biotin-labeled cRNA, produced by in vitro transcription, was fragmented and hybridized to the Affymetrix U133A GeneChip arrays at 45° C. for 16 hrs and then washed and stained using the GeneChip Fluidics. The arrays were scanned by a GeneArray Scanner and patterns of hybridization were detected as light emitted from the fluorescent reporter groups incorporated into the target and hybridized to oligonucleotide probes. Full details of the methods used for RNA extraction and development of gene expression data from lung and ovarian tumors have been described previously. (Bild A, Yao G, Chang J T, et al: Oncogenic pathways signatures in human cancers as guide to targeted therapies. Nature 439(7074):353-357, 200, Potti A, Dressman H K, Bild A, et al: Genomic signatures to guide the use of chemotherapeutics. Nature Medicine 12(11): 1294-1300, 2006).

In one embodiment, determining the expression level (or obtaining a first gene expression profile) of multiple genes in a tumor sample from the subject comprises extracting a nucleic acid sample from the sample from the subject. In certain embodiments, the nucleic acid sample is an mRNA sample. In one embodiment, the expression level of the nucleic acid is determined by hybridizing the nucleic acid, or amplification products thereof, to a DNA microarray. Amplification products may be generated, for example, with reverse transcription, optionally followed by PCR amplification of the products.

### (C) Genes Screened

In one embodiment, the predictive methods of the invention comprise determining the expression level of all the genes in the cluster that define at least one therapeutic sensitivity/resistance determinative metagene. In one embodiment, the predictive methods of the invention comprise determining the expression level of at least 50%, 60%, 70%, 80%, 90%, 95%, 98%, 99% of the genes in each of the clusters that defines 1 or 2 or more therapeutic sensitivity/resistance determinative metagenes. A metagene is a cluster or set of genes which may be used to predict sensitivity or resistance to a therapeutic agent.

In one embodiment, at least 50%, 60%, 70%, 80%, 90%, 95%, 98%, 99% of the genes whose expression levels are used in order to predict cisplatin sensitivity (or the genes in the cluster that define a metagene having said predictivity) are genes listed in Table 1. In one embodiment, at least 50%, 60%, 70%, 80%, 90%, 95%, 98%, 99% of the genes whose expression levels are used in order to predict pemetrexed sensitivity are genes listed in Table 2. In one embodiment, at least 50%, 60%, 70%, 80%, 90%, 95%, 98%, 99% of the genes whose expression levels are determined to predict sensitivity to either cisplatin or pemetrexed (or the genes in the cluster that define a metagene having said predictivity) are genes listed in Table 1 or Table 2.

In one embodiment, at least 50%, 60%, 70%, 80%, 90%, 95%, 98%, 99% of the genes listed in Table 1 are used to predict responsiveness of a cancer to a platinum based chemotherapeutic agent, such as cisplatin. In one embodiment, at least 50%, 60%, 70%, 80%, 90%, 95%, 98%, 99% of the genes listed in Table 2 are used to predict responsiveness of a cancer to an antimetabolite chemotherapeutic agent, such as pemetrexed. In one embodiment, at least 50%, 60%, 70%, 80%, 90%, 95%, 98%, 99% of the genes listed in Table 1 or Table 2 are used to predict responsiveness of a cancer to a platinum-based or antimetabolite chemotherapeutic agent, such as cisplatin or pemetrexed.

Tables 1 and 2 show the genes in the cluster that define metagenes 1 and 2 and indicate the therapeutic agent whose sensitivity it predicts. In one embodiment, at least 3, 5, 7, 9, 10, 12, 14, 16, 18, 20, 25, 30, 40 or 50 genes in the cluster of genes defining a metagene used in the methods described herein are common to metagene 1 or 2, or to both 1 and 2.

### (D) Metagene Valuation

In one embodiment, the predictive methods of the invention comprise defining the value of one or more metagenes from the expression levels of the genes. A metagene value is defined by extracting a single dominant value from a cluster of genes associated with sensitivity to an anti-cancer agent.

In one embodiment, the dominant single value is obtained using single value decomposition (SVD). In one embodiment, the cluster of genes of each metagene or at least of one metagene comprises at least 3, 4, 5, 6, 7, 8, 9, 10, 12, 15, 18, 20 or 25 genes.

In one embodiment, the predictive methods of the invention comprise defining the value of at least one metagene wherein the genes in the cluster of genes from which the metagene is defined, shares at least 50%, 60%, 70%, 80%, 90%, 95% or 98% of genes in common to anyone of metagenes 1 or 2. In one embodiment, the predictive methods of the invention comprise defining the value of at least two metagenes, wherein the genes in the cluster of genes from which each metagene is detined share at least 50%, 60%, 70%, 80%, 90%, 95% or 98% of genes in common to anyone of metagenes 1 or 2. In one embodiment, the predictive methods of the invention comprise defining the value of a metagene from a cluster of genes, wherein at least 2, 3, 4, 5, 6, 7, 8, 9, 10, 11, 12, 13, 14, 15, 16, 17, 18, 19 or 20 genes in the cluster are selected from the genes listed in Tables 1 or 2.

In one embodiment, the clusters of genes that define each metagene are identified using supervised classification methods of analysis as previously described. See, for example, West, M. et al. Proc Natl Acad Sci USA 98, 11462-11467 (2001). The analysis selects a set of genes whose expression levels are most highly correlated with the classification of tumor samples into sensitivity to an anti-cancer agent versus no sensitivity to an anti-cancer agent. The dominant principal components from such a set of genes then defines a relevant phenotype-related metagene, and regression models, such as binary regression models, assign the relative probability of sensitivity to an anti-cancer agent.

(E) Predictions from Tree Models

In one embodiment, the predictive methods of the invention comprise averaging the predictions of one or more statistical tree models applied to the metagene values, wherein each model includes one or more nodes, each node representing a metagene, each node including a statistical predictive probability of sensitivity to an anti-cancer agent. The statistical tree models may be generated using the methods described herein for the generation of tree models. General methods of generating tree models may also be found in the art (See for example Pitman et al, Biostatistics 2004; 5:587-601; Denison et al. Biometrika 1999; 85:363-77; Nevins et al. Hum Mol Genet 2003; 12:R153-7; Huang et al. Lancet 2003; 361: 1590-6; West et al. Proc Natl A cad Sci USA 2001; 98:11462-7; U.S. Patent Pub. Nos. 2003-0224383; 2004-0083084; 2005-0170528; 2004-0106113; and U.S. application Ser. No. 11/198782).

In one embodiment, the predictive methods of the invention comprise deriving a prediction from a single statistical tree model, wherein the model includes one or more nodes, each node representing a metagene, each node including a statistical predictive probability of sensitivity to an anti-cancer agent. In alternative embodiments, the tree may comprise at least 2, 3, 4, or 5 nodes.

In one embodiment, the predictive methods of the invention comprise averaging the predictions of one or more statistical tree models applied to the metagene values, wherein each model includes one or more nodes, each node representing a metagene, each node including a statistical predictive probability of sensitivity to an anti-cancer agent. Accordingly, the invention provides methods that use mixed trees, where a tree may contain at least two nodes, where each node represents a metagene representative of the sensitivity/resistance to a particular agent.

In one embodiment, the statistical predictive probability is derived from a Bayesian analysis. In another embodiment, the Bayesian analysis includes a sequence of Bayes factor based tests of association to rank and select predictors that define a node binary split, the binary split including a predictor/threshold pair. Bayesian analysis is an approach to statistical analysis that is based on the Bayes law, which states that the posterior probability of a parameter p is proportional to the prior probability of parameter p multiplied by the likelihood of p derived from the data collected. This methodology represents an alternative to the traditional (or frequentist probability) approach: whereas the latter attempts to establish confidence intervals around parameters, and/or falsify a-priori null-hypotheses, the Bayesian approach attempts to keep track of how a priori expectations about some phenomenon of interest can be refined, and how observed data can be integrated with such a priori beliefs, to arrive at updated posterior expectations about the phenomenon. Bayesian analysis has been applied to numerous statistical models to predict outcomes of events based on available data. These include standard regression models, e.g. binary regression models, as well as to more complex models that are applicable to multi-variate and essentially non-linear data.

Another such model is commonly known as the tree model which is essentially based on a decision tree. Decision trees can be used in clarification, prediction and regression. A decision tree model is built starting with a root mode, and training data partitioned to what are essentially the “children” nodes using a splitting rule. For instance, for clarification, training data contains sample vectors that have one or more measurement variables and one variable that determines that class of the sample. Various splitting rules may be used. A statistical predictive tree model to which Bayesian analysis is applied may consistently deliver accurate results with high predictive capabilities.

Gene expression signatures that reflect the activity of a given pathway may be identified using supervised classification methods of analysis previously described (e.g., West, M. et al. Proc Natl Acad Sci USA 98, 11462-11467, 2001). The analysis selects a set of genes whose expression levels are most highly correlated with the classification of tumor samples into sensitivity to an anti-cancer agent versus no sensitivity to an anti-cancer agent. The dominant principal components from such a set of genes then defines a relevant phenotype-related metagene, and regression models assign the relative probability of sensitivity to an anti-cancer agent.

In one embodiment, the each statistical tree model generated by the methods described herein comprises 2, 3, 4, 5, 6 or more nodes. In one embodiment of the methods described herein for defining a statistical tree model predictive of sensitivity/resistance to a therapeutic, the resulting model predicts cancer sensitivity to an anti-cancer agent with at least 70%, 80%, 85%, or 90% or higher accuracy. In another embodiment, the model predicts sensitivity to an anti-cancer agent with greater accuracy than clinical variables. In one embodiment, the clinical variables are selected from age of the subject, gender of the subject, tumor size of the sample, stage of cancer disease, histological subtype of the sample and smoking history of the subject. In one embodiment, the cluster of genes that define each metagene comprise at least 3, 4, 5, 6, 7, 8, 9, 10, 12 or 15 genes. In one embodiment, the correlation-based clustering is Markov chain correlation-based clustering or K-means clustering.

### Gene Chips and Kits

Arrays and microarrays which contain the gene expression profiles for determining responsivity to platinum-based therapy, pemetrexed-based therapy, and/or responsivity to salvage agents are also encompassed within the scope of this invention. Methods of making arrays are well-known in the art and as such do not need to be described in detail here.

Such arrays can contain the profiles of 5, 10, 15, 20, 25, 30, 40, 50, 75, 100, 150, 200 or more genes as disclosed in the Tables. Accordingly, arrays for detection of responsivity to particular therapeutic agents can be customized for diagnosis or treatment of specific cancers, such as ovarian cancer, breast cancer, or NSCLC. The array can be packaged as part of kit comprising the customized array itself and a set of instructions for how to use the array to determine an individual's responsivity to a specific cancer therapeutic agent.

Also provided are reagents and kits thereof for practicing one or more of the above described methods. The subject reagents and kits thereof may vary greatly. Reagents of interest include reagents specifically designed for use in production of the above described metagene values.

One type of such reagent is an array probe of nucleic acids, such as a DNA chip, in which the genes defining the metagenes in the therapeutic efficacy predictive tree models are represented. A variety of different array formats are known in the art, with a wide variety of different probe structures, substrate compositions and attachment technologies. Representative array structures of interest include those described in U.S., Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; as well as WO 95/21265; WO 96/31622; WO 97/10365; WO 97/27317; EP 373 203; and EP 785 280; the disclosures of which are herein incorporated by reference.

The DNA chip is conveniently used to compare the expression levels of a number of genes at the same time. DNA chip-based expression profiling can be carried out, for example, by the method as disclosed in “Microarray Biochip Technology” (Mark Schena, Eaton Publishing, 2000). A DNA chip comprises immobilized high-density probes to detect a number of genes. Thus, the expression levels of many genes can be estimated at the same time by a single-round analysis. Namely, the expression profile of a specimen can be determined with a DNA chip. A DNA chip may comprise probes, which have been spotted thereon, to detect the expression level of the metagene-defining genes of the present invention, i.e. the genes described in Tables 1 and 2. A probe may be designed for each marker gene selected, and spotted on a DNA chip. Such a probe may be, for example, an oligonucleotide comprising 5-50 nucleotide residues. Methods for synthesizing such oligonucleotides on DNA chips are known to those skilled in the art. Longer DNAs can be synthesized by PCR or chemically. Methods for spotting long DNA, which is synthesized by PCR or the like, onto a glass slide are also known to those skilled in the art. A DNA chip that is obtained by the methods described above can be used for estimating the efficacy of a therapeutic agent in treating a subject afflicted with cancer according to the present invention.

DNA microarray and methods of analyzing data from microarrays are well-described in the art, including in DNA Microarrays: A Molecular Cloning Manual. Ed. by Bowtel and Sambrook (Cold Spring Harbor Laboratory Press, 2002); Microarrays for an Integrative Genomics by Kohana (MIT Press, 2002); A Biologist's Guide to Analysis of DNA Micraarray Data, by Knudsen (Wiley, John & Sons, Incorporated, 2002); DNA Microarrays: A Practical Approach, Vol. 205 by Schema (Oxford University Press, 1999); and Methods of Microarray Data Analysis II, ed. by Lin et al. (Kluwer Academic Publishers, 2002) all of which are incorporated herein by reference.

One aspect of the invention provides a kit comprising: (a) any of the gene chips described herein; and (b) one of the computer-readable mediums described herein.

In some embodiments, the arrays include probes for at least 2, 3, 4, 5, 6, 7, 8, 9, 10, 15, 20, 25, 30, 40, or 50 of the genes listed in Table 1 or Table 2. In certain embodiments, the number of genes that are from Table 1 that are represented on the array is at least 5, at least 10, at least 25, at least 50, at least 75 or more, including all of the genes listed in the table. In certain embodiments, the number of genes that are from Table 2 that are represented on the array is at least 5, at least 10, at least 25, at least 50, at least 75 or more, including all of the genes listed in the table. Where the subject arrays include probes for additional genes not listed in the tables, in certain embodiments the number % of additional genes that are represented does not exceed about 50%, 40%, 30%, 20%, 15%, 10%, 8%, 6%, 5%, 4%, 3%, 2% or 1%. In some embodiments, a great majority of genes in the collection are genes that define the metagenes of the invention, whereby great majority is meant at least about 75%, usually at least about 80% and sometimes at least about 85, 90, 95% or higher, including embodiments where 100% of the genes in the collection are metagene-defining genes. In an alternative embodiment, the arrays for use in the invention may include a majority of probes that are not listed in Table 1 or Table 2.

The kits of the subject invention may include the above described arrays or gene chips. The kits may further include one or more additional reagents employed in the various methods, such as primers for generating target nucleic acids, dNTPs and/or rNTPs, which may be either premixed or separate, one or more uniquely labeled dNTPs and/or rNTPs, such as biotinylated or Cy3 or Cy5 tagged dNTPs, gold or silver particles with different scattering spectra, or other post synthesis labeling reagent, such as chemically active derivatives of fluorescent dyes, enzymes, such as reverse transcriptases, DNA polymerases, RNA polymerases, and the like, various buffer mediums, e.g. hybridization and washing buffers, prefabricated probe arrays, labeled probe purification reagents and components, like spin columns, etc., signal generation and detection reagents, e,g. streptavidin-alkaline phosphatase conjugate, chemifluorescent or chemiluminescent substrate, and the like.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a remote site. Any convenient means of conveying instructions may be present in the kits.

The kits also include packaging material such as, but not limited to, ice, dry ice, styrofoam, foam, plastic, cellophane, shrink wrap, bubble wrap, paper, cardboard, starch peanuts, twist ties, metal clips, metal cans, drierite, glass, and rubber.

### Computer Readable Media Comprising Gene Expression Profiles

The invention also contemplates computer readable media that comprises gene expression profiles. Such media can contain all or part of the gene expression profiles of the genes listed in the Tables that comprise the responsivity predictor set. The media can be a list of the genes or contain the raw data for running a user's own statistical calculation, such as the methods disclosed herein.

Another aspect of the invention provides a program product (i.e., software product) for use in a computer device that executes program instructions recorded in a computer-readable medium to perform one or more steps of the methods described herein, such for estimating the efficacy of a therapeutic agent in treating a subject afflicted with cancer.

One aspect of the invention provides a computer readable medium having computer readable program codes embodied therein, the computer readable medium program codes performing one or more of the following functions: defining the value of one or more metagenes from the expression levels of genes in known responsive and sensitive cells; defining a metagene value by extracting a single dominant value using singular value decomposition (SVD) from a cluster of genes associated with tumor sensitivity to a therapeutic agent; averaging the predictions of one or more statistical tree models applied to the values of the metagenes; or averaging the predictions of one or more binary regression models applied to the values of the metagenes, wherein each model includes a statistical predictive probability of tumor sensitivity to a therapeutic agent.

Another related aspect of the invention provides kits comprising the program product or the computer readable medium, optionally with a computer system. One aspect of the invention provides a system, the system comprising: a computer; a computer readable medium, operatively coupled to the computer, the computer readable medium program codes performing one or more of the following functions: defining the value of one or more metagenes from the expression levels genes; defining a metagene value by extracting a single dominant value using singular value decomposition (SVD) from a cluster of genes associated tumor sensitivity to a therapeutic agent; averaging the predictions of one or more statistical tree models applied to the values of the metagenes; or averaging the predictions of one or more binary regression models applied to the values of the metagenes, wherein each model includes a statistical predictive probability of tumor sensitivity to a therapeutic agent.

In one embodiment, the program product comprises: a recordable medium; and a plurality of computer-readable instructions executable by the computer device to analyze data from the array hybridization steps, to transmit array hybridization from one location to another, or to evaluate genome-wide location data between two or more genomes. Computer readable media include, but are not limited to, CD-ROM disks (CD-R, CD-RW), DVD-RAM disks, DVD-RW disks, floppy disks and magnetic tape.

A related aspect of the invention provides kits comprising the program products described herein. The kits may also optionally contain paper and/or computer-readable format instructions and/or information, such as, but not limited to, information on DNA microarrays, on tutorials, on experimental procedures, on reagents, on related products, on available experimental data, on using kits, on chemotherapeutic agents including their toxicity, and on other information. The kits optionally also contain in paper and/or computer-readable format information on minimum hardware requirements and instructions for running and/or installing the software. The kits optionally also include, in a paper and/or computer readable format, information on the manufacturers, warranty information, availability of additional software, technical services information, and purchasing information. The kits optionally include a video or other viewable medium or a link to a viewable format on the internet or a network that depicts the use of the use of the software, and/or use of the kits. The kits also include packaging material such as, but not limited to, styrofoam, foam, plastic, cellophane, shrink wrap, bubble wrap, paper, cardboard, starch peanuts, twist ties, metal clips, metal cans, drierite, glass, and rubber.

The analysis of data, as well as the transmission of data steps, can be implemented by the use of one or more computer systems. Computer systems are readily available. The processing that provides the displaying and analysis of image data for example, can be performed on multiple computers or can be performed by a single, integrated computer or any variation thereof. The components contained in the computer system are those typically found in general purpose computer systems used as servers, workstations, personal computers, network terminals, and the like. In fact, these components are intended to represent a broad category of such computer components that are well known in the art.

The following examples are provided to illustrate aspects of the invention but are not intended to limit the invention in any manner.

### Examples

### Example 1

### Development and Characterization of Platinum Chemotherapy Responsivity Predictor Set

The purpose of this study was to develop new genomic-based tools for personalized treatment of patients with advanced-stage cancer. The inventors have utilized gene expression profiles to identify patients likely to be resistant to primary platinum-based chemotherapy and also to identify alternate targeted therapeutic options for patients with de-novo platinum resistant disease. The inventors had previously developed a platinum-based therapy predictor set comprising 100 genes (US20070172844). The new platinum-based therapy predictor set described herein is an improvement on the previous predictor set because it achieves superior sensitivity (100% vs 83%) and similar accuracy (83.1% vs 84.3%) compared to the previous predictor set. In addition, the new predictor set comprises only 45 genes compared to the 100 genes of the previous predictor set, significantly increasing the ease and speed of use while reducing the cost. Furthermore, the previous predictor set and new predictor set were derived differently; the previous predictor set was derived from clinical specimens with data from patient response to chemotherapeutic drugs, while the new predictor set was derived from cell lines grown in vitro and assayed for drug resistance in vitro. Surprisingly, this methodology provided better sensitivity without loss of accuracy using a smaller set of predictors.

### Material And Methods

In vitro chemosensitivity predictors. The [−log10(M)] GI50/IC50 and LC50 (50% cytotoxic dose) data on the NCI-60 cell line panel for cisplatin was used to populate a matrix with MATLAB software with the relevant expression data for each individual cell line. When multiple entries for a drug screen existed (by NCS number), the entry with the largest number of replicates was included. To develop an in vitro gene expression based predictor of cisplatin sensitivity from the phalmacologic data used in the NCI-60 drug screen studies, we chose cell lines within the NCl-60 panel that would represent the extremes of sensitivity (The NCI-60 cell lines and the sensitivity data are available on the internet at dtp.nci.nih.gov/docs/cancer/cancer data.html). Our hypothesis was that such a selection would identify cell lines that represent the extremes of sensitivity to a given drug (Potti A, Dressman H K, Bild A, et al: Genomic signatures to guide the use of chemotherapeutics. Nature Medicine 12(11): 1294-1300, 2006). Relevant expression data (updated data available on the Affymetrix U95A2 GeneChip) for the selected NCI-60 cell lines were then used in a supervised analysis using Bayesian regression methodologies, as described previously (Pittman J, Huang E, Nevins J, et al: Bayesian analysis of binary prediction tree models for retrospectively sampled outcomes. Biostatistics 5(4): 587-601, 2004), to develop a probit model predictive of sensitivity to cisplatin.

The collection of data in the NCI-60 data did not represent a significant diversity in resistant and sensitive cell lines to cisplatin. Thus, if a drug screening experiment did not result in widely variable GI50/IC50 and/or LC50 data, the generation of a genomic predictor is not possible using our methods, as in the case of cisplatin. Thus, we used data published by (Gyorffy B, et al: Gene expression profiling of 30 cancer cell lines predicts resistance towards 11 anticancer drugs at clinically achieved concentrations. Int J Cancer 118(7): 1699-1712, 2005, which is incorporated herein by reference in its entirety), where they had determined definitive resistance and sensitivity to cisplatin in 30 cancer cell lines. Importantly, we also had access to corresponding gene expression data to facilitate the generation of a model which would predict sensitivity to cisplatin. The cell names and an indication of sensitivity or resistance to cisplatin are in Table 3. All array data are available on the supplemental website (data.cgt.duke.edu/JCO.php).

Statistical analysis methods. Analysis of expression data was performed as previously described (Potti A, Dressman H K, Bild A, et al: Genomic signatures to guide the use of chemotherapeutics. Nature Medicine 12(11): 1294-1300, 2006). Prior to statistical modeling, gene expression data was filtered to exclude probe sets with signals present at background noise levels, and for probe sets that did not vary significantly across samples. Each signature summarizes its constituent genes as a single expression profile, and was here derived as the top principal components of that set of genes. When predicting the chemosensitivity patterns of cancer cell lines or tumor samples, gene selection and identification was based on the training data (i.e. the cell lines with known sensitivity or resistance to cisplatin), and then metagene values were computed using the principal components of the training data and additional cell line or tumor expression data. Bayesian fitting of binary probit regression models to the training data then permitted an assessment of the relevance of the metagene signatures in within-sample classification (Berridge M V and Tan A S: Characterization of the cellular reduction of 3-(4,5-dimethylthiazol-2-yl)-2,5-diphenyl tetrazolium bromide (MTT): subcellular localization, substrate dependence, and involvement if mitochondrial electron transport in MTT reduction. Arch Biochem Biophys 303(2):474-482, 1993), and estimation and uncertainty assessments for the binary regression weights mapping metagenes to probabilities.

To guard against over-fitting given the disproportionate number of variables to samples, we also performed leave-one-out cross validation analysis to test the stability and predictive capability of our model. Each sample was left out of the data set one at a time, the model was refitted (both the metagene factors and the partitions used) using the remaining samples, and the phenotype of the held out case was then predicted and the certainty of the classification was calculated. Given a training set of expression vectors (of values across metagenes) representing two biological states, a binary probit regression model, of predictive probabilities for each of the two states (resistant vs. sensitive) was estimated using Bayesian methods for each case. Predictions of the relative chemosensitivity of the validation cell lines or tumor samples were then evaluated using methods previously described (Berridge M V and Tan A S: Characterization of the cellular reduction of 3-(4,5-dimethylthiazol-2-yl)-2,5-diphenyltetrazolium bromide (MTT): subcellular localization, substrate dependence, and involvement if mitochondrial electron transport in MTT reduction. Arch Biochem Biophys 303(2):474-482, 1993, Bild A, Yao G, Chang J T, et al: Oncogenic pathways signatures in human cancers as guide to targeted therapies. Nature 439(7074):353-357, 2006) producing estimated relative probabilities of chemosensitivity across the validation set. Probabilities were scaled from 0 to 1 with a score of >0.5 representing the cutoff of being in the selected state.

The statistical analysis involved in generating predictive models indicative of chemotherapeutic sensitivity used standard binary regression models combined with singular value decompositions SVDs, also referred to as singular factor decompositions, and with stochastic regularization using Bayesian analysis. It is beyond the scope here to provide full technical details, so the interested reader is referred to manuscripts referred to above.

Some key details are elaborated here. Assume n tumors and p genes, and write X for the p×n matrix of expression values, with rows as genes and columns as tumors. Column i of X is the p-vector X of expression levels of all genes on tumor i. Singular factor decomposition of the set of expression measures on the sample of tumors has the form X=ADF, where D is a diagonal n×n matrix of non-negative singular values, A is a p'n matrix with orthogonal columns, and F is an n×n orthonormal matrix of (metagene) factor Values. Column i of F, denoted by f, is the n-vector of values of all n factors on tumor i, and we have xᵢ=ADfᵢ. In the current study as an example, write p(x) for the probability tumor/cell line i is chemosensitive versus chemoresistant. A probit regression sets p(xᵢ)=P(b¹x₁) where P is the standard normal distribution function and b¹x; is a linear combination of expression levels based on the p-vector of regression parameters b. Then p(x₁)=F(g¹f₁) where g=DA¹b is a n-vector of regression coefficients for the factors. Hence regression on genes reduces to regression on “metagene” factors, a much lower dimensional inference problem.

The resulting Bayesian analysis may be easily implemented using standard iterative Markov chain Monte Carlo (MCMC) simulation methods of Bayesian analysis to impute sets of simulated parameter values whose distributions are summarized to produce point and interval estimates of model parameters g as well as of probabilities of chemotherapy sensitivity in both the training set and validation samples. This involves the standard method of imputing the latent normal variates implicit in the probit function as part of the simulation analysis. The orthogonality of the factor design implied by the orthonormality of F leads to the use of independent Student T prior distributions on the elements of the factor regression parameter vector g and model fitting involves representing the T distributions as scale mixtures of normal priors and includes estimation of the implicit scale factors in the MCMC analysis, again a standard technique. Analyses reported are based on Student T priors with 2 degrees of freedom, providing relatively vague prior forms.

In addition to posterior samples for the factor parameters g, the MCMC approach leads directly to the calculations required for prediction of chemotherapy sensitivity for any given predictor. Most importantly, the Bayesian SVD regression framework allows direct inversion to infer the parameters b from g, to provide inferences about which genes are important in defining p(x), and how subsets of genes interact. Specifically, new theory shows that the relevant inversion is simply the least-norm generalized inverse b=AD¹ g. Hence posterior sample values for g are trivially mapped to corresponding sample values of b, and summarized to produce posterior estimates of b.

It is pertinent to explore comparisons of the chosen binary regression, using the probit form, with alternatives such as the standard logistic. We have done this, making repeat analysis using models in which P is a Student T distribution rather than normal, and with varying the degrees of freedom within which the Student T with 8 or 9 degrees of freedom very closely approximates the standard logistic function. Following, the MCMC analysis of the probit is trivially extended to Student T models. In these studies, predictive results and interpretation of the cell line and tumor response data are not altered significantly, indicating robustness to the assumed form in this setting.

Cell and RNA preparation. Full details of the methods used for RNA extraction and development of gene expression data from lung and ovarian tumors have been described previously (Potti A, Dressman H K, Bild A, et al: Genomic signatures to guide the use of chemotherapeutics. Nature Medicine 12(11):1294-1300, 2006). Briefly, total RNA was extracted using the Qiashredder and Qiagen RNeasy Mini kit and the quality of RNA was checked by an Agilent 2100 Bioanalyzer. The targets for Affymetrix DNA microarray analysis were prepared according to the manufacturer's instructions. Biotin-labeled cRNA, produced by in vitro transcription, was fragmented and hybridrized to the Affymetrix U133A GeneChip arrays at 45° C. for 16 hrs and then washed and stained using GeneChip Fluidics. The arrays were scanned by a GeneArray Scanner and patterns of hybridization were detected as light emitted from the fluorescent reporter groups incorporated into the target and hybridized to oligonucleotide probes. All analyses were performed in a MIAME (minimal information about a microarray experiment)-compliant fashion, as defined in the guidelines established by MGED.

Classification of Platinum response in ovarian tumors. Using Affymetrix U133A GeneChips, we measured gene expression in 59 patients with advanced (FlGO stage III/IV) serous epithelial ovarian carcinomas who received cisplatin therapy (GEO accession number: GSE3149). All ovarian cancer specimens were obtained at initial cytoreductive surgery from patients.

Response to therapy was evaluated using standard criteria for patients with measurable disease, based upon WHO guidelines (Therasse P, Arbuck S G, Eisenhauer E A, et al: New guidelines to evaluate the response to treatment in solid tumors. European organization for research and treatment of cancer, National cancer institute of the US, National cancer institute of Canada. J Natl Cancer Inst 92(3):205-216, 2000). CA-125 was used to classify responses only in the absence of a measurable lesion and based on established guidelines (Rustin G J, Timmers P, Nelstrop A. et al: Companson of CA-125 and standard definitions of progression of ovarian cancer in the intergroup trial of cisplatin and paditaxel versus cisplatin and cyclophosphamide. J Clin Oneal. 24(I):45-51,2006). A complete response (CR) was defined as a complete disappearance of all measurable and assessable disease or, in the absence of measurable lesions, a normalization of the CA-125 level following therapy. A partial response (PR) was considered a 50% or greater reduction in the product obtained from measurement of each bi-dimensional lesion for at least 4 weeks or a drop in the CA-125 by at least 50% for at least 4 weeks. Progressive disease (PD) was defined as a 50% or greater increase in the product from any lesion documented within 8 weeks of initiation of therapy, appearance of any new lesion within 8 weeks of initiation of therapy, or a doubling of CA-125 from baseline. For the purposes of our analysis, a clinically beneficial response (i.e. “responder”) included CR or PR. A patient who did not demonstrate a CR or PR was considered a “non-responder”.

Cross-platform Affymetrix Gene Chip comparison. To map the probe sets across various generations of Affymetrix GeneChip arrays, we utilized Chip Comparex as described previously (Bild A, Yao G, Chang. J T, et al: Oncogenic pathways signatures in human cancers as guide to targeted therapies, Nature 439(7074):353-357,2006, Potti A, Oressmall H K, Bild A, et al: Genomic signatures to guide the use of chemotherapeutics. Nature Medicine 12(II): 1294-1300, 2006, which is incorporated herein by reference in its entirety).

Lung and ovarian cancer cell culture. The NSCLC cell lines (H23, H225, H322, H358, H441, H520, A549, H647, H838, H1650, H1651, H1666, H1734, H1793, H1838, H2030 and H2 126) were grown as recommended by the supplier (ATCC, Rockville, Mo.). The ovarian cancer cell lines (OCV AR-3, OVCAR-5, TOV-21G and TOV-1120) were grown as recommended by the supplier (ATCC, Rockville, Mo.). FUOV-1, a human ovarian carcinoma, was grown according to the supplier (DSMZ, Braunschweig, Germany). Ten additional cell lines (C13, OV2008, A2780-CP, A2780S, IGROV-1, T8, IMCC3, 1MCC5, SKOV3 and A200S) were provided by Dr. Patricia Kruk (University of South Florida, Fla.). These ten cell lines were grown in RPMI 1640, supplemented with 10% Fetal Bovine Serum, 1% Sodium pyruvate, and 1% non essential amino acids. Tissue culture media and Thiazolyl Blue Tetrazolium Bromide were purchased from Sigma Aldrich (St. Louis, Mo.). Cisplatin and Pemetrexed were obtained from the pharmacy at Duke Medical Center.

Cell proliferation and Drug sensitivity assays. Optimal cell number and linear range of drug concentration were determined for each cell line and drug as described previously (Bild A, Yao G, Chang S T, et al: Oncogenic pathways signatures in human cancers as guide to targeted therapies. Nature 439(7074):353-357, 2006, Potti A, Dressman H K, Bild A, et al: Genomic signatures to guide the use of chemotherapeutics. Nature Medicine 12(11): 1294-1300, 2006). For drug sensitivity assay, cells were plated in non drug containing media in 96-well plates. After incubating for 24 hrs at 37° C., drugs were added to each well at a specific concentration. Cells were grown in the presence of drugs for an additional 96 hrs and sensitivity to cisplatin, docetaxel, paclitaxel, and pemetrexed in the cell lines was determined by quantifying the percent reduction in growth (versus DMSO controls) at 96 hrs using a standard MTT colorimetric assay (CellTiter 96 Aqueous One 23 Solution Cell Proliferation Assay Kit (Promega)) (Mosmann T: Rapid Colorimetric Assay for Cellular Growth and Survival: Application to proliferation and cytotoxic assay. J Immunol Meth. 65(1-2):55-63, 1983, Berridge M V and Tan A S: Characterization of the cellular reduction of 3-(4,5-dimethylthiazol-2-yl)-2,5-diphenyltetrazolium bromide (MTT): subcellular localization, substrate dependence, and involvement if mitochondrial electron transport in MTT reduction. Arch Biochem Biophys 303(2):474-482, 1993). All experiments were repeated in triplicate.

### Developing a Gene Expression-Based Predictor of Cisplatin Sensitivity

The experimental strategy for analysis employed in this study is similar to that used for the development of oncogenic pathway and chemotherapy sensitivity signatures as described previously (Bild A, et al: Oncogenic pathways signatures in human cancers as guide to targeted therapies. Nature 439(7074):353-357, 2006, Potti A, et al: Genomic signatures to guide the use of chemotherapeutics. Nature Medicine 12(11): 1294-1300, 2006). Samples representing extreme cases were used to train the expression data to develop a genomic signature that can predict drug sensitivity. A predictor of cisplatin sensitivity was developed by analyzing cell lines described by Gyorffy el at. (Gyorffy R et al: Gene expression profiling of 30 cancer cell lines predicts resistance towards 11 anticancer drugs at clinically achieved concentrations. Int J Cancer 118(7): 1699-1712, 2005).

Using Bayesian binary regression analysis, genes highly correlated with drug sensitivity were identified and used to develop a model that could differentiate between cisplatin sensitivity and resistance. The developed model consisting of 45 genes based on cisplatin sensitivity (FIG. 1a) was validated in a leave-one-out cross validation. The cisplatin sensitivity predictor includes DNA repair genes such as ERCCJ and ERCC4 among others that had altered expression in the list of cisplatin sensitivity predictor genes. (Table 1). Interestingly, one previously described mechanism of resistance to cisplatin therapy is due to the increased capacity of cancer cells to repair DNA damage incurred, by activation of DNA repair genes (Johnson S, Perez R, Godwin A, et al, Role of platinum-DNA adduct formation and removal in cisplatin resislance in human ovarian cancer cell lines. Biochem Pharmacol 47:689-697, 1994, Yen L, Woo A, Christopoulopoulos G, et al., Enhanced host cell reactivation capacity and expression of DNA repair genes in human breast cancer cells resistant to bi-functional alkylating agents. Mutation Research 337; 179-189, 1995).

### In Vitro Validation of the Cisplatin Predictor

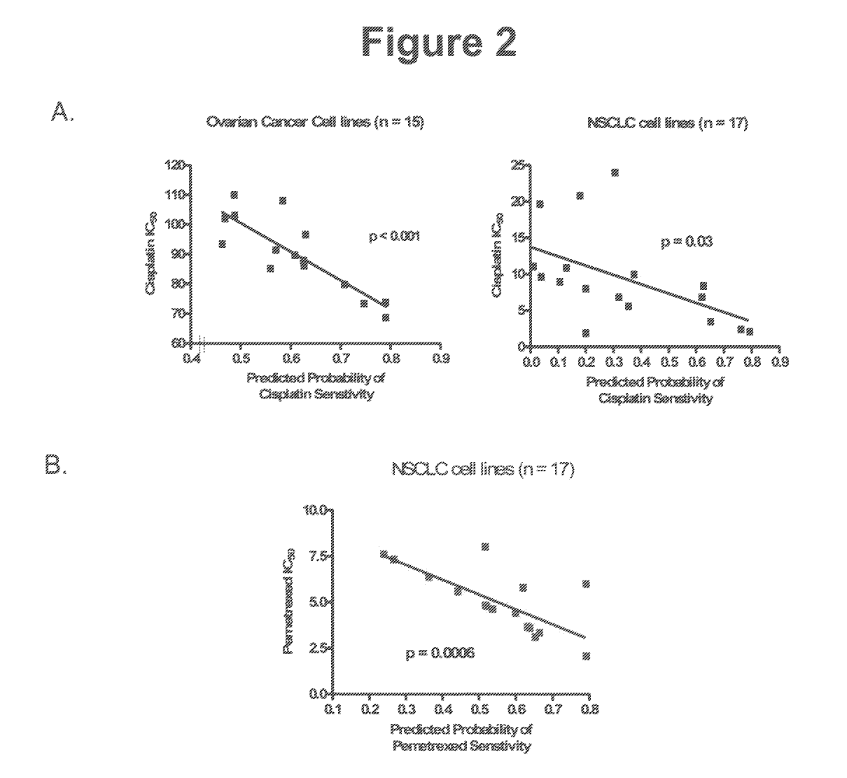

In addition to initial leave-one-out cross validation, the true value of a predictor lies in its ability to predict sensitivity in independent in vitro and in vivo settings. In the present study, the predictor of cisplatin sensitivity was independently validated in a panel of 32 (lung and ovarian cancer) cell lines, using cell proliferation assays and concurrent gene expression data. As shown in FIG. 2a, the correlation between the predicted probability of sensitivity to cisplatin (in both lung and ovarian cell lines) and the respective IC50 for cisplatin confirmed the capacity of the cisplatin predictor set to accurately predict sensitivity to the drug in cancer cell lines.

### In Vivo Validation of the Cisplatin Sensitivity Predictor.

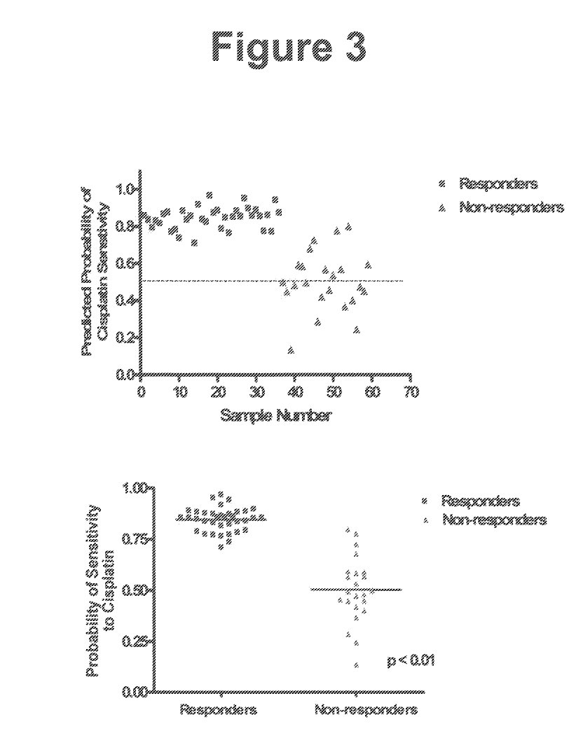

Although the ability of the cisplatin signature to predict sensitivity in independent samples validates the performance of the signature, it is the ability to predict response in patients that is obviously most critical. Using data from a previously published study that linked gene expression data with clinical response to cisplatin in an ovarian data set (Bild A, Yao G, Chang. J T, et al: Oncogenic pathways signatures in human cancers as guide to targeted therapies. Nature 439(7074):353-357, 2006) (GEO accession number: GSE3149), we tested the ability of the in vitro cisplatin sensitivity predictor to accurately identify those patients that responded to cisplatin. Using a predicted probability of response of 0.50 as the cut-off for predicting cisplatin sensitivity, the accuracy of the in vitro gene expression-based predictor of cisplatin sensitivity, based on available clinical data, was 83.1% (Sensitivity 100%, Specificity 57%, PPV 78%, NPV 100%) (FIG. 3). Furthermore, a Mann-Whitney U-test revealed a significant difference in the predicted probabilities of cisplatin sensitivity between the resistant and sensitive cohorts of patients (p<0.01) (FIG. 3).

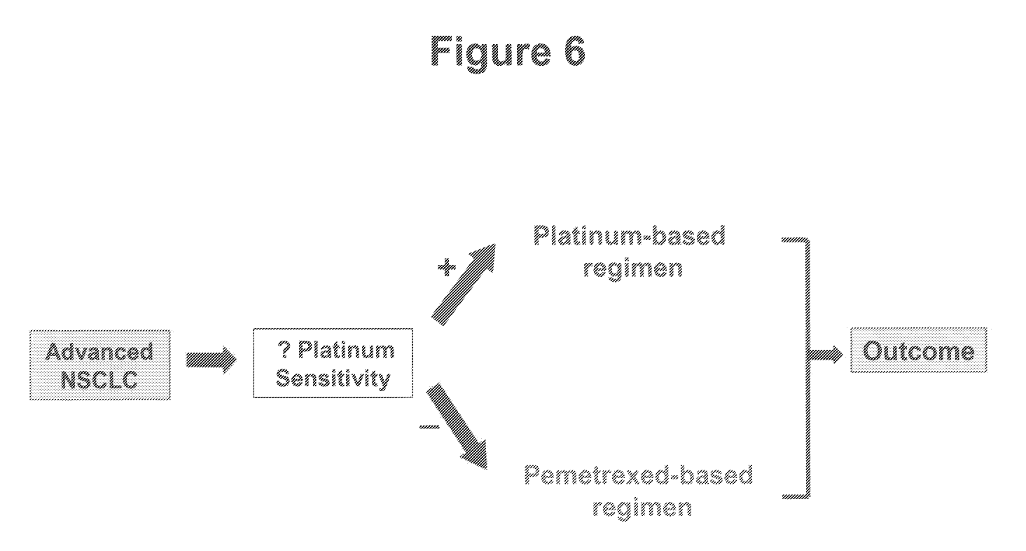

In this study, the patterns of cisplatin sensitivity observed in our cohort of 91 NSCLC tumors suggests that not all patients may initially respond to first line cisplatin based therapy. As described above, response rates to first-line platinum based therapy is around 30% with median survival between 24 to 31 months (Breathnach O S, Freidlin B, Conley B, et al: Twenty-two years of phase III trials for patients with advanced non-small-cell lung cancer: sobering results. J Clin OncoI 19(6): 1734-42, 2001). We have made use of in vitro drug sensitivity data in cancer cell lines, coupled with Affymetrix expression data, to develop gene expression signatures reflecting sensitivity to cisplatin and pemetrexed. The capacity of these signatures to predict response in independent sets of cell lines and patient studies begins to define a strategy that addresses the potential to identify cytotoxic agents that best match individual patients with advanced NSCLC and other advanced cancers (ovarian cancer). In addition, it can potentially be applied to patients with early-stage NSCLC to predict who may benefit from adjuvant cisplatin-based therapy. The performance of a genomic signature-based selection of a chemotherapy agent as an initial step in the individualized treatment strategy for patients with advanced cancer would be useful (FIG. 6).

### Example 2

### Development and Characterization of Gene Expression Profiles that Determine Response to Pemetrexed Chemotherapy for Ovarian Cancer and NSCLC

### Material And Methods

Gene expression predictor sets for response to pemetrexed were determined much as gene expression predictor sets for response to platinum-based therapy as described in Example 1. Since pemetrexed is a member of the class of antimetabolite chemotherapeutic agents which function through common mechanisms, it is likely that a given cancer will respond similarly to pemetrexed and other antimetabolites. Therefore the gene expression predictor set for response to pemetrexed is likely to also predict sensitivity to other antimetabolites.

In vitro chemosensitivity predictors. The [-log10(M)] GI50/IC50 and LC50 (50% cytotoxic dose) data on the NCI-60 cell line panel for pemetrexed was used to populate a matrix with MATLAB software with the relevant expression data for each individual cell line. When multiple entries for a drug screen existed (by NCS number), the entry with the largest number of replicates was included. To develop an in vitro gene expression based predictor of pemetrexed sensitivity from the phalmacologic data used in the NCI-60 drug screen studies, we chose cell lines within the NCl-60 panel that would represent the extremes of sensitivity (The NCI-60 cell lines and the sensitivity data are available on the internet at dtp.nci.nih.gov/docs/cancer/cancer data.html). Our hypothesis was that such a selection would identify cell lines that represent the extremes of sensitivity to a given drug (Potti A, Dressman H K, Bild A, et al: Genomic signatures to guide the use of chemotherapeutics. Nature Medicine 12(11): 1294-1300, 2006). Relevant expression data (updated data available on the Affymetrix U95A2 GeneChip) for the selected NCI-60 cell lines were then used in a supervised analysis using Bayesian regression methodologies, as described previously (Pittman J, Huang E, Nevins J, et al: Bayesian analysis of binary prediction tree models for retrospectively sampled outcomes. Biostatistics 5(4): 587-601, 2004), to develop a probit model predictive of sensitivity to pemetrexed. See Table 4 for the cell line information.

Lung and ovarian cancer cell culture. The NSCLC cell lines (H23, H225, H322, H358, H441, H520, A549, H647, H83g, H1650, H1651, H1666, H1734, H1793, H1838, H2030 and H2126) were grown as recommended by the supplier (ATCC, Rockville, Md.). The ovarian cancer cell lines (OCV AR-3, OVCAR-5, TOV-21 G and TOV-112D) were grown as recommended by the supplier (ATCC, Rockville, Md.). FUOV-1, a human ovarian carcinoma, was grown according to the supplier (DSMZ, Braunschweig, Germany). Ten additional cell lines (C13, OV2008, A2780-CP, A2780S, IGROV-1, T8, IMCC3, IMCC5, SKOV3 and A2008) were provided by Dr. Patricia Kruk (University of South Florida, Fla.). These ten cell lines were grown in RPMI 1640, supplemented with 10% Fetal Bovine Serum, 1% Sodium pyruvate, and 1% non essential amino acids. Tissue culture media and Thiazolyl Blue Tetrazolium Bromide were purchased from Sigma Aldrich (St. Louis, Mo.). Cisplatin and Pemetrexed were obtained from the pharmacy at Duke Medical Center.

Cell proliferation and Drug sensitivity assays. Optimal cell number and linear range of drug concentration were determined for each cell line and drug as described previously (Bild A, Yao G, Chang J T, et al: Oncogenic pathways signatures in human cancers as guide to targeted therapies. Nature 4:19(7074):353-357, 2006, Potti A, Oressman H K, Bild A, et al: Genomic signatures to guide the use of chemotherapeutics. Nature Medicine 12(11): 1294-1300, 2006). For drug sensitivity assay, cells were plated in non drug containing media in 96-well plates. After incubating for 24 hrs at 37° C., drugs were added to each well at a specific concentration. Cells were grown in the presence of drugs for an additional 96 hrs and sensitivity to cisplatin, docetaxel, pacliraxel, and pemetrexed in the cell lines was determined by quantifying the percent reduction in growth (versus DMSO controls) at 96 hrs using a standard MTT colorimetric assay (CellTiter 96 Aqueous One 23 Solution Cell Proliferation Assay Kit (Promega)) (Mosmann T: Rapid Colorimetric Assay for Cellular Growth and Survival: Application to prolifemtion and cytotoxic assay. J Immunol Meth. 65(1-2):55-63,1983, Berridge M V and Tan A S: Characterization of the cellular reduction of 3-(4,5-dimethylthiazol-2-yl)-2,5-diphenyltetrazolium bromide (MTT): subcellular localization, substrate dependence, and involvement if mitochondrial electron transport in MTT reduction. Arch Biochem Biophys 303(2):474-482, 1993). All experiments were repeated in triplicate.

### Results

The major motivation for this study was the characterization of the genomic basis of ovarian cancer and NSCLC response to pemetrexed chemotherapy. We present a preliminary predictive tool that may identify patients most likely to benefit from pemetrexed therapy for recurrent or persistent cancer at the time of initial diagnosis. Further, by defining the oncogenic pathways that contribute to pemetrexed resistance we hope to identify additional therapeutic options for patients predicted to have cancer resistant to single-agent pemetrexed therapy.

### Developing a Gene Expression-Based Predictor of Pemetrexed Sensitivity

In NSCLC, where platinum-based therapy is the standard of care, response rates are only 30%. One approach to identifying potential drugs effective in cisplatin-resistant patients, is to examine the NCI-60 dataset for agents whose IC50 profile showed an inverse relationship with cisplatin, focusing on those known to be effective in NSCLC. Of these drugs, an inverse correlation with cisplatin sensitivity was identified with docetaxel, abraxane and pemetrexed. The strongest inverse correlation was found between cisplatin and pemetrexed sensitivity (p<0.001; Pearson r value: 0.1; alpha: 0.05).

Using methods previously described (Potti A, Dressman H K, Bild A, et al: Genomic signatures to guide the use of chemotherapeutics. Nature Medicine 12(11): 1294-1300,2006), a predictor of pemetrexed sensitivity was developed by identifying NCI-60 cell lines that were most resistant or sensitive to pemetrexed. Using Bayesian binary regression analysis, genes whose expression was most highly correlated with drug sensitivity were used to develop a predictive model that could differentiate between pemetrexed sensitivity and resistance. The developed model consisting of 85 genes based on pemetrexed sensitivity (FIG. 1b) was validated in a leave-one-out cross validation.

### In Vitro Validation of the Cisplatin and Pemetrexed Predictor

Similar to the independent validation of the cisplatin sensitivity predictor, the pemetrexed predictor was validated using gene expression data from an independent cohort of 17 NSCLC cell lines with respective in vitro drug sensitivity assays. As shown in FIG. 2b, the correlation between the predicted probability of sensitivity to pemetrexed in the 17 NSCLC cell lines and the respective IC50 for pemetrexed validated the ability of the pemetrexed predictor to predict sensitivity to the drug in an independent cohort of cancer cell lines.

### Patterns of Predicted Chemotherapy Response to Cisplatin and Pemetrexed in NSCLC

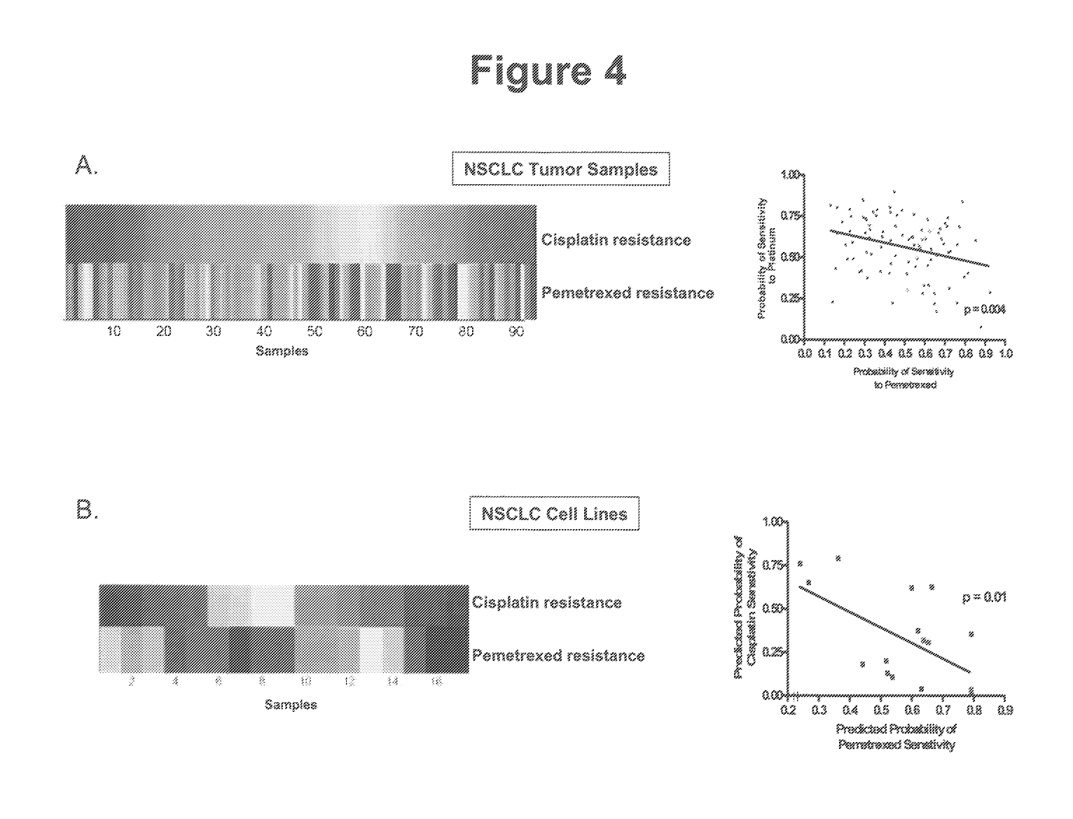

The cisplatin and pemetrexed predictors were utilized to profile the potential options of using these two drugs in a collection of 91 NSCLC described previously (Potti A, Mukherjee S, Petersen R, et al., A Genomic Strategy to Refine Prognosis in Early-Stage Non-Small-Cell Lung Cancer. NEJM 355:570-80, 2006) (GEO accession number: GSE3141). These samples were first sorted according to the patterns of predicted sensitivity to cisplatin (FIG. 4a, left panel). The pattern observed indicated that those patients resistant to cisplatin (red) were more sensitive to pemetrexed (blue). Although the data points in the scatter plot do not appear to be perfectly correlated, this analysis suggests that the relationship was statistically significant (p=0.004, log rank) (FIG. 4a, right panel). A similar relationship was also demonstrated in the independent cohort of NSCLC cell lines (FIG. 4b) suggesting the possibility of an alternative therapy for treatment of advanced or metastatic NSCLC patients who would be predicted to be platinum resistant. As a comparison, the pemetrexed signature was also applied to the ovarian cancer patient data set. In this analysis however, only 2/59(<1%) patients were identified to have greater than 50% probability of being sensitive to pemetrexed.

The Sequence of chemotherapy May be Critical in Optimizing Responses.

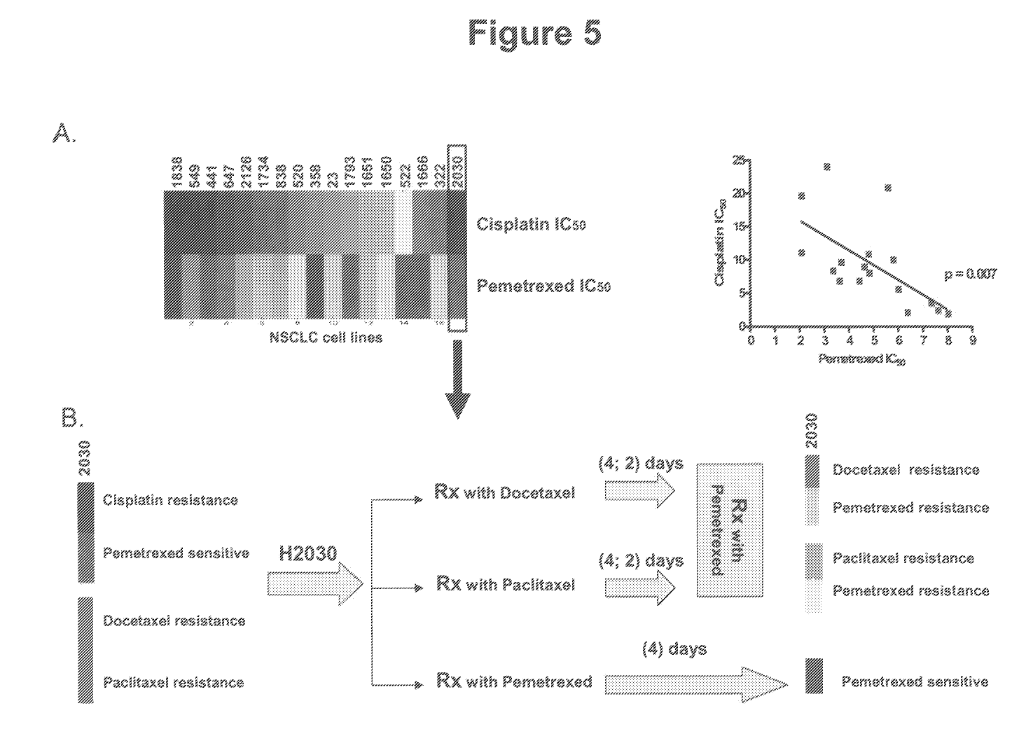

Currently, first-line treatment with a platinum-based regimen is the standard of care for advanced NSCLC. Those patients developing resistance to cisplatin are treated with a taxane, pemetrexed, or erlotinib as second line options. To explore the effect of cisplatin resistance, as well as prior treatment with potentially ineffective therapies, the IC5O of various lung cancer cell lines to cisplatin and pemetrexed were analyzed and revealed an inverse relationship (FIG. 5a). Thereafter, one NSCLC cell line (H2030) that is resistant to cisplatin, paclitaxel, and docetaxel, but sensitive to pemetrexed, based on cell proliferation assays (IC50) was treated with pemetrexed, docetaxel, or paclitaxel in a systematic fashion. Interestingly, when H2030 was first treated for four days with a taxane (docetaxel or paclitaxel), resistance to subsequent pemetrexed exposure was induced (FIG. 5b). In contrast, when H2030 was first treated with pemetrexed, H2030 was sensitive, as expected (FIG. 5b).

Although these in vitro observations are only hypothesis generating at this time, this proof of principle experiment suggests that the sequence of second line chemotherapy in NSCLC may prove to be important in determining clinical outcomes. Specifically, tumors from cisplatin refractory patients who are also predicted to be resistant to a taxane, when treated with a taxane (docetaxel or paclitaxel) prior to pemetrexed therapy may induce resistance to subsequent pemetrexed therapy. This suggests the importance of including genomic-based, disease specific, treatment prioritization in clinical practice.

### REFERENCE BIBLIOGRAPHY

1. Levin L, Simon R, Hryniuk W: Importance of multiagent chemotherapy regimens in ovarian carcinoma: dose intensity analysis. J. Natl. Cane. Inst. 85:1732-1742, 1993.
2. McGuire W P, Hoskins W J, Brady M F, et al: Assessment of dose-intensive therapy in suboptimally debulked ovarian cancer; a Gynecologic Oncology Group study. J Clin. Oncol. 13:1589-1599, 1995.
3. Jodrell D I, Egorin M J, Canetta R M, et al: Relationships between carboplatin exposure and tumor response and toxicity in patients with ovarian cancer, J Clin. Oncol. 10:520-528, 1992.
4. McGuire W P, Hoskins W J, Brady M F, et al: Cyclophosphamide and cisplatin compared with paclitaxel and cisplatin in patients with stage III and stage IV ovarian cancer. N Engl. J. Med. 334:1-6, 1996.
5. McGuire W P, Brady M F, Ozols R F: The Gynecologic Oncology Group experience in ovarian cancer. Ann Oncol. 10:29-34, 1999.
6. Piccart M J, Bertelsen K, Stuart G, et al: Long-term follow-up confirms a survival advantage of the paclitaxel-cisplatin regimen over the cyclophosphamide-cisplatin combination in advanced ovarian cancer. Int. J. Gynecol Cancer 13:144-148, 2003.
7. Wenham R M, Lancaster J M, Berchuck A: Molecular aspects of ovarian cancer. Best Pract. Res. Clin. Obstet. Gynecol. 16:483-497, 2002.
8. Berchuck A, Kohler M F, Marks J R, el al: The p53 tumor suppressor gene frequently is altered in gynecologic cancers. Am. J. Obstet. Gynecol. 170:246-252, 1994.
9. Kohler M F, Marks J R, Wiseman R W, et al: Spectrum of mutation and frequency of aJlelic deletion of the p53 gene in ovarian cancer. J. Natl. Cane. Inst. 85:1513-1519, 1993.
10. Havrilesky L, Alvarez A A, Whitaker R S, et al: Loss of expression of the p 16 tumor suppressor gene is more frequent in advanced ovarian cancers lacking p53 mutations. Gynecol. Oncol. 83:491-500, 2001.
11. Rel es A, Wen W H, Schmider A, et al: Correlation of p53 mutations with resistance to platinum-based chemotherapy and shortened survival in ovarian cancer. Clinical Cancer Research 7:29R84-2997, 2001.
12. Schmider A, Gee C, Friedmann W, et al: p21 (WAF1/CIPI) protein expression is associated with prolonged survival but not with p53 expression in epithelial ovarian carcinoma. Gynecol, Oncol. 77:237-242, 2000.
13. Wong K K, Cheng R S, Mok S C: identification of differentially expressed genes from ovarian cancer cells by MICROMAX cDNA microarray system. Biotechniques 30:670-675, 2001.
14. Welsh J B, Zarrinkar P P, Sapinoso L M, et al: Analysis of gene expression profiles in nonnal and neoplastic ovarian tissue samples identifies candidate molecular markers of epithelial ovarian cancer. Proc. Natl. Acad. Sci. USA 98: 1176-1181, 2001.
15. Shridhar V, Lee J-S, Pandita A, et al: Genetic analysis of early-versus late-state ovarian tumors. Cancer Res. 61:5895-5904, 2001.
16. Schummer M, Ng W W, Bumgarner R E, et al: Comparative hybridization of an array of 21,500 ovarian cDNAs for the discovery of genes overexpressed in ovarian carcinomas. Gene 238:375-385,1999.
17. Ono K, Tanaka T, Tsunoda T, et al: Identification by cDNA microarray of genes involved in ovarian carcinogenesis. Cancer Res. 60:5007-5011, 2000.
18. Sawiris G P, Sherman-Baust C A, Becker K G, et al: Development of a highly specialized cDNA array for the study and diagnosis of epithelial ovarian cancer. Cancer Res. 62:2923-2928, 2002.
19. Jazaeri A A, Yee C J, Sotiriou C, et al: Gene expression profiles of BRCA1-linked, BRCA2-linked, and sporadic ovarian cancers. J Natl. Cane. Inst. 94:990-1000, 2002.
20. Schaner M E, Ross D T, Ciaravino G, et al: Gene expression patterns in ovarian carcinomas. Mol. Biol. Cell 14:4376-4386, 2003.
21. Lancaster J M, Dressman H, Whitaker R S, et al: Gene expression patterns that characterize advanced stage serous ovarian cancers. J. Surgical Gynecol. Invest. 11:51-59, 2004.
22. Berchuck A, Iversen E S, Lancaster J M, et al: Patterns of gene expression that characterize long term survival in advanced serous ovarian cancers. Clin. Can. Res. 11:3686-3696, 2005.
23. Berchuck A, Iversen E, Lancaster J M, et al: Prediction of optimal versus suboptimal cytoreduction of advanced stage serous ovarian cancer using microarrays. Am. J. Obstet. Gynecol. 190:910-925, 2004.
24. Jazaeri A A, Awtrey Cs, Chandramouli G V, et al: Gene expression profiles associated with response to chemotherapy in epithelial ovarian cancers. Clin. Cancer Res. 11:6300-6310, 2005.
25. Helleman J, Jansen M P, Span P N, et al: Molecular profiling of platinum resistant ovarian cancer. Int. J. Cancer 118:1963-1971, 2005.
26. Spentzos D, Levine D A, Kolia s, et al: Unique gene expression profile based on pathologic response in epithelial ovarian cancer. J. Clin. Oncol. 23:7911-7918, 2005.
27. Spentzos D, Levine D A, Ramoni M F, et al: Gene expression signature with independent prognostic significance in epithelial ovarian cancer. J. Clin. Oncol. 22:4700-4710, 2004.
28. Miller A B, Hoogstraten B. Staquet I\:f, et al: Reporting results of cancer treatment. Cancer 47:207-214, 1981.
29. Rustin G J, Nelstrop A E, Bentzen S M, et al : Use of tumor markers in monitoring the course of ovarian cancer. Ann. Oncol. 10:21-27, 1999.
30. Rustin G J, Nelstrop A E, McClean P, et al: Defining response of ovarian carcinoma to initial chemotherapy according to serum CA 125. J. Clin. Oncol. 14:1 545-1551, 1996.
31. Irizarry R A, Hobbs B, Collin F, et al: Exploration, normalization, and summaries of high density oligonucleotide array probe level data. Biostatistics 4:249-263, 2003.
32. Bolstad B M, Irizarry R A. Astrand M, et al: A comparison of normalization methods for high density oligonucleotide array data based on variance and bias. Bioinformatics 19:185-193, 2003.
33. Lucus J, Carvalho C, Wang Q, et al: Sparse statistical modeling in gene expression genomics. Cambridge, Cambridge University Press, 2006.
34. Rich J, Jones B, Hans C, et al: Gene expression profiling and genetic markers in glioblastoma survival. Cancer Res. 65:4051-4058, 2005.
35. Hans C, Dobra A, West M: Shotgun stochastic search for regression with many candidate predictors. JASA in press., 2006.
36. Bild A, Yao G, Chang J T, et al: Oncogenic pathway signatures in human cancers as a guide to targeted therapies. Nature 439:353-357, 2006.
37. Gyorrfy B, Surowiak P, Kiesslich O, Denkert C, Schafer R, Dietel M, Lage H: Gene expression profiling of 30 cancer cell lines predicts resistance towards 11 anticancer drugs at clinically achieved concentrations. Int. J. Cancer 118(7):1699-712, 2006.
38. Minna, J D, Gazdar, A F, Sprang, S R & Herz, J: Cancer. A bull's eye for targeted lung cancer therapy. Science 304:1458-1461, 2004.
39. Jemal et al., CA Cancer J. Clin., 53, 5-26, 2003.
40. Cancer Facts and Figures: American Cancer Society, Atlanta, p. 11, 2002.
41. Travis et al., Lung Cancer Principles and Practice, Lippincott-Raven, New York, pps. 361-395, 1996.
42. Gazdar et al., Anticancer Res. 14:261-267.
43. Niklinska et al., Folia Histochem. Cytobiol. 39:147-148, 2001.
44. Parker et al, CA Cancer J. Clin. 47:5-27, 1997.
45. Chu et al, J. Nat. Cancer Inst. 88:1571-1579, 1996.
46. Baker, V V: Salvage therapy for recurrent epithelial ovarian cancer. Hematol. Oncol. Clin. N. Am. 17:977-988, 2003.
47. Hansen, H H, Eisenhauer, E A, Hasen M, Neijt I P, Piccart M I, Sessa C, Thigpen J T: New cytostatis drugs in ovarian cancer. Ann. Oncol. 4:S63-S70, 1993.
48. Herrin, V E, Thigpen I T: Chemotherapy for ovarian cancer: current concepts. Semin. Surg. Oncol. 17:181-188, 1999.
49. Staunton, J. E. et al. Chemosensitivity prediction by transcriptional profiling. Proc Natl Acad Sci USA 98:10787-19792, 2001.
50. Chang, J. C. et al. Gene expression profiling for the prediction of therapeutic response to docetaxel in patients with breast cancer. Lancet 362:362-369, 2003.
51. Emi, M., Kim, R., Tanabe, K., Uchida, y, & toge, T. Targeted therapy against Bcl-2-related proteins in breast cancer cells. Breast Cancer Res 7: R940-R952, 2005.
52. Takahashi, T. et al. Cyclin A-associated kinase activity is needed for paclitaxel sensitivity. Mol Cancer Ther 4: 1039-1046, 2005.
53. Modi, S. et al. Phosphorylated/activated HER2 as a marker of clinical resistance to single agent taxane chemotherapy for metastatic breast cancer. Cancer Invest 23:483-487, 2005.
54. Langer, R. et at Association of pretherapeutic expression of chemotherapy-related genes with response to neoadjuvant chemotherapy in Barrett carcinoma. Clin Cancer Res. 11:7462-7469, 2005.
55. Rouzier, R. et at., Breast cancer molecular subtypes respond differently to preoperative chemotherapy. Clin Cancer Res. 11:5678-5685, 2005.
56. Rouzier, R. et at Microbubule-associated protein tau: a marker of paclitaxel sensitivity on breast cancer. Proc Natl Acad Set USA 102:8315-8320, 2005.
57. DeVita, V. T., Hellman, S. & Rosenberg, S. A. Cancer: Principles and Practice of Oncology, Lippincott-Raven, Philadelphia, 2005.
58. Herbst, R. S. et al. Clinical Cancer Advances 2005; Major research advances in cancer treatment, prevention, and screening—a report from the American Society of Clinical Oncology. J Clin. Oncol. 24:190-205, 2006.
59. Broxterman, H. J. & Georgopapadakou, N. H. Anticancer therapeutics: Addictive targets, multi-targeted drugs, new drug combinations. Drug Resist Update 8:183-197, 2005.
60. Pittman, J., Huang, E., Wang, Q., Nevins, J. R. & West, M. Bayesian analysis of binary prediction tree models for retrospectively sampled outcomes. Biostatistics 5:587-601, 2004.
61. West, M. et al. Predicting the clinical status of human breast cancer by using gene expression profiles. Proc Natl Acad Sci USA 98:11462-11467, 2001.
62. Ihaka, R. & Gentleman, R. A language for data analysis and graphics. J Comput. Graph. Stat. 5:299-314, 1996.
63. Eisen, M. B., Spellman, P T, Brown, P. O. & Botstein, D. Cluster analysis and display of genome-wide expression patterns. Proc Natl Acad Sci USA 95:14863-14868, 1998.
64. Patti A, Dressman H K, Bild A, et al: Genomic signatures to guide the use of chemotherapeutics. Nature Medicine 12(11): 1294-1300, 2006.

TABLE 1
Cisplatin Responsivity Predictor Set

| | | Entrez | | | | |
|---|---|---|---|---|---|---|
| Cisplatin | Probe Set | Gene | Representative | Gene | Go biological | |
| weight | ID | ID | Public ID | Symbol | process term | Go molecular function term |
| 0.141112 | 200076_s_at | 79036 | BC006479 | C19orf50 | — | protein binding |
| −0.528138 | 200711_s_at | 6500 | NM_003197 | SKP1 | ubiquitin- | protein binding /// protein |
| | | | | | dependent protein | binding |
| | | | | | catabolic process | |
| | | | | | /// ubiquitin cycle | |
| 1.527462 | 200719_at | 6500 | BE964043 | SKP1 | ubiquitin- | protein binding /// protein |
| | | | | | dependent protein | binding |
| | | | | | catabolic process | |
| | | | | | /// ubiquitin cycle | |
| −0.396061 | 201200_at | 8804 | NM_003851 | CREG1 | regulation of cell | RNA polymerase II |
| | | | | | growth /// | transcription factor activity /// |
| | | | | | regulation of | transcription corepressor |
| | | | | | transcription from | activity |
| | | | | | RNA polymerase | |
| | | | | | II promoter /// | |
| | | | | | multicellular | |
| | | | | | organismal | |
| | | | | | development /// | |
| | | | | | cell proliferation | |
| 0.478106 | 201375_s_at | 5516 | NM_004156 | PPP2CB | protein amino | phosphoprotein phosphatase |
| | | | | | acid | activity /// protein |
| | | | | | dephosphorylation | serine/threonine phosphatase |
| | | | | | | activity /// iron ion binding /// |
| | | | | | | protein binding /// protein |
| | | | | | | binding /// hydrolase activity |
| | | | | | | /// manganese ion binding /// |
| | | | | | | metal ion binding /// protein |
| | | | | | | heterodimerization activity |
| 1.440668 | 201422_at | 10437 | NM_006332 | IFI30 /// | signal | protein binding /// protein |
| | | /// | | PIK3R2 | transduction /// | binding /// 1- |
| | | 5296 | | | negative | phosphatidylinositol-3-kinase |
| | | | | | regulation of anti- | activity /// 1- |
| | | | | | apoptosis /// | phosphatidylinositol-3-kinase |
| | | | | | negative | activity /// oxidoreductase |
| | | | | | regulation of anti- | activity /// phosphoinositide 3- |
| | | | | | apoptosis | kinase regulator activity |
| −0.287759 | 202005_at | 6768 | NM_021978 | ST14 | proteolysis /// | catalytic activity /// serine- |
| | | | | | proteolysis | type endopeptidase activity /// |
| | | | | | | peptidase activity /// serine- |
| | | | | | | type peptidase activity /// |
| | | | | | | hydrolase activity |
| 0.986002 | 202016_at | 4232 | NM_002402 | MEST | mesoderm | catalytic activity /// protein |
| | | | | | development | binding |
| 0.603289 | 202329_at | 1445 | NM_004383 | CSK | protein amino | nucleotide binding /// protein |
| | | | | | acid | kinase activity /// protein |
| | | | | | phosphorylation | tyrosine kinase activity /// |
| | | | | | /// protein amino | protein tyrosine kinase activity |
| | | | | | acid | /// non-membrane spanning |
| | | | | | phosphorylation | protein tyrosine kinase activity |
| | | | | | /// negative | /// protein binding /// protein |
| | | | | | regulation of cell | binding /// ATP binding /// |
| | | | | | proliferation | protein C-terminus binding /// |
| | | | | | | kinase activity /// transferase |
| | | | | | | activity |
| 3.572925 | 202889_x_at | 9053 | T62571 | MAP7 | microtubule | structural molecule activity |
| | | | | | cytoskeleton | |
| | | | | | organization and | |
| | | | | | biogenesis /// | |
| | | | | | establishment | |
| | | | | | and/or | |
| | | | | | maintenance of | |
| | | | | | cell polarity | |
| 1.172302 | 203021_at | 6590 | NM_003064 | SLPI | — | endopeptidase inhibitor |
| | | | | | | activity /// endopeptidase |
| | | | | | | inhibitor activity /// serine- |
| | | | | | | type endopeptidase inhibitor |
| | | | | | | activity /// peptidase activity |
| | | | | | | /// protease inhibitor activity |
| −0.607092 | 203119_at | 79080 | NM_024098 | CCDC86 | — | — |
| 0.53851 | 203126_at | 3613 | NM_014214 | IMPA2 | phosphate | magnesium ion binding /// |
| | | | | | metabolic process | inositol or |
| | | | | | /// signal | phosphatidylinositol |
| | | | | | transduction | phosphatase activity /// |
| | | | | | | inositol-1(or 4)- |
| | | | | | | monophosphatase activity /// |
| | | | | | | inositol-1(or 4)- |
| | | | | | | monophosphatase activity /// |
| | | | | | | hydrolase activity /// metal ion |
| | | | | | | binding |
| −0.270294 | 203638_s_at | 2263 | NM_022969 | FGFR2 | protein amino | nucleotide binding /// protein |
| | | | | | acid | kinase activity /// protein |
| | | | | | phosphorylation | tyrosine kinase activity /// |
| | | | | | /// protein amino | protein tyrosine kinase activity |
| | | | | | acid | /// transmembrane receptor |
| | | | | | phosphorylation | protein tyrosine kinase activity |
| | | | | | /// cell growth | /// receptor activity /// |
| | | | | | | fibroblast growth factor |
| | | | | | | receptor activity /// protein |
| | | | | | | binding /// ATP binding /// |
| | | | | | | heparin binding /// kinase |
| | | | | | | activity /// transferase activity |
| −2.299763 | 203639_s_at | 2263 | M80634 | FGFR2 | protein amino | nucleotide binding /// protein |
| | | | | | acid | kinase activity /// protein |
| | | | | | phosphorylation | tyrosine kinase activity /// |
| | | | | | /// protein amino | protein tyrosine kinase activity |
| | | | | | acid | /// transmembrane receptor |
| | | | | | phosphorylation | protein tyrosine kinase activity |
| | | | | | /// cell growth | /// receptor activity /// |
| | | | | | | fibroblast growth factor |
| | | | | | | receptor activity /// protein |
| | | | | | | binding /// ATP binding /// |
| | | | | | | heparin binding /// kinase |
| | | | | | | activity /// transferase activity |
| 0.140491 | 203701_s_at | 55621 | NM_017722 | TRMT1 | tRNA processing | nucleic acid binding /// RNA |
| | | | | | | binding /// tRNA (guanine- |
| | | | | | | N2-)-methyltransferase |
| | | | | | | activity /// methyltransferase |
| | | | | | | activity /// zinc ion binding /// |
| | | | | | | transferase activity |
| 1.785986 | 203729_at | 2014 | NM_001425 | EMP3 | multicellular | — |
| | | | | | organismal | |
| | | | | | development /// | |
| | | | | | cell death /// | |
| | | | | | negative | |
| | | | | | regulation of cell | |
| | | | | | proliferation /// | |
| | | | | | cell growth | |
| 1.566223 | 203973_s_at | 1052 | NM_005195 | CEBPD | transcription /// | DNA binding /// transcription |
| | | | | | regulation of | factor activity /// sequence- |
| | | | | | transcription, | specific DNA binding /// |
| | | | | | DNA-dependent | protein dimerization activity |
| | | | | | /// transcription | |
| | | | | | from RNA | |
| | | | | | polymerase II | |
| | | | | | promoter | |
| 0.840481 | 204437_s_at | 2348 | NM_016725 | FOLR1 | receptor-mediated | receptor activity /// receptor |
| | | | | | endocytosis /// | activity /// folic acid binding |
| | | | | | folic acid | /// folic acid binding |
| | | | | | transport /// folic | |
| | | | | | acid metabolic | |
| | | | | | process | |
| 2.444132 | 205037_at | 11020 | NM_006860 | RABL4 | small GTPase | nucleotide binding /// GTP |
| | | | | | mediated signal | binding |
| | | | | | transduction | |
| 2.298408 | 205822_s_at | 3157 | NM_002130 | HMGCS1 | lipid metabolic | catalytic activity /// |
| | | | | | process /// steroid | hydroxymethylglutaryl-CoA |
| | | | | | biosynthetic | synthase activity /// |
| | | | | | process /// | hydroxymethylglutaryl-CoA |
| | | | | | cholesterol | synthase activity /// transferase |
| | | | | | biosynthetic | activity |
| | | | | | process /// | |
| | | | | | metabolic process | |
| | | | | | /// isoprenoid | |
| | | | | | biosynthetic | |
| | | | | | process /// lipid | |
| | | | | | biosynthetic | |
| | | | | | process /// sterol | |
| | | | | | biosynthetic | |
| | | | | | process | |
| 0.637989 | 207076_s_at | 445 | NM_000050 | ASS1 | urea cycle /// urea | nucleotide binding /// |
| | | | | | cycle /// arginine | argininosuccinate synthase |
| | | | | | biosynthetic | activity /// protein binding /// |
| | | | | | process /// amino | ATP binding /// ligase activity |
| | | | | | acid biosynthetic | |
| | | | | | process | |
| 0.124318 | 207746_at | 10721 | NM_014125 | POLQ | DNA replication | nucleotide binding /// nucleic |
| | | | | | /// DNA repair /// | acid binding /// DNA binding |
| | | | | | DNA repair /// | /// damaged DNA binding /// |
| | | | | | response to DNA | DNA-directed DNA |
| | | | | | damage stimulus | polymerase activity /// DNA- |
| | | | | | | directed DNA polymerase |
| | | | | | | activity /// helicase activity /// |
| | | | | | | ATP binding /// ATP- |
| | | | | | | dependent helicase activity /// |
| | | | | | | transferase activity /// |
| | | | | | | nucleotidyltransferase activity |
| | | | | | | /// hydrolase activity |
| 0.449295 | 207761_s_at | 25840 | NM_014033 | METTL7A | metabolic process | methyltransferase activity /// |
| | | | | | | transferase activity |
| −0.504686 | 208228_s_at | 2263 | M87771 | FGFR2 | protein amino | nucleotide binding /// protein |
| | | | | | acid | kinase activity /// protein |
| | | | | | phosphorylation | tyrosine kinase activity /// |
| | | | | | /// protein amino | protein tyrosine kinase activity |
| | | | | | acid | /// transmembrane receptor |
| | | | | | phosphorylation | protein tyrosine kinase activity |
| | | | | | /// cell growth | /// receptor activity /// |
| | | | | | | fibroblast growth factor |
| | | | | | | receptor activity /// protein |
| | | | | | | binding /// ATP binding /// |
| | | | | | | heparin binding /// kinase |
| | | | | | | activity /// transferase activity |
| 0.8175 | 208791_at | 1191 | M25915 | CLU | lipid metabolic | protein binding |
| | | | | | process /// | |
| | | | | | apoptosis /// anti- | |
| | | | | | apoptosis /// | |
| | | | | | immune response | |
| | | | | | /// complement | |
| | | | | | activation /// | |
| | | | | | complement | |
| | | | | | activation, | |
| | | | | | classical pathway | |
| | | | | | /// response to | |
| | | | | | oxidative stress /// | |
| | | | | | cell death /// | |
| | | | | | positive | |
| | | | | | regulation of cell | |
| | | | | | proliferation /// | |
| | | | | | endocrine | |
| | | | | | pancreas | |
| | | | | | development /// | |
| | | | | | innate immune | |
| | | | | | response /// | |
| | | | | | positive | |
| | | | | | regulation of cell | |
| | | | | | differentiation /// | |
| | | | | | neurite | |
| | | | | | morphogenesis | |
| 2.105379 | 209771_x_at | 934 | AA761181 | CD24 | response to | signal transducer activity /// |
| | | | | | hypoxia /// cell | protein binding /// protein |
| | | | | | activation /// | binding /// protein kinase |
| | | | | | regulation of | binding /// carbohydrate |
| | | | | | cytokine and | binding /// protein tyrosine |
| | | | | | chemokine | kinase activator activity |
| | | | | | mediated | |
| | | | | | signaling pathway | |
| | | | | | /// regulation of | |
| | | | | | cytokine and | |
| | | | | | chemokine | |
| | | | | | mediated | |
| | | | | | signaling pathway | |
| | | | | | /// response to | |
| | | | | | molecule of | |
| | | | | | bacterial origin /// | |
| | | | | | response to | |
| | | | | | molecule of | |
| | | | | | bacterial origin /// | |
| | | | | | immune response- | |
| | | | | | regulating cell | |
| | | | | | surface receptor | |
| | | | | | signaling pathway | |
| | | | | | /// elevation of | |
| | | | | | cytosolic calcium | |
| | | | | | ion concentration | |
| | | | | | /// neuromuscular | |
| | | | | | synaptic | |
| | | | | | transmission /// | |
| | | | | | induction of | |
| | | | | | apoptosis by | |
| | | | | | intracellular | |
| | | | | | signals /// Wnt | |
| | | | | | receptor signaling | |
| | | | | | pathway /// cell- | |
| | | | | | cell adhesion /// | |
| | | | | | cell migration /// | |
| | | | | | cell migration /// | |
| | | | | | regulation of | |
| | | | | | epithelial cell | |
| | | | | | differentiation /// | |
| | | | | | T cell | |
| | | | | | costimulation /// B | |
| | | | | | cell receptor | |
| | | | | | transport into | |
| | | | | | membrane raft /// | |
| | | | | | chemokine | |
| | | | | | receptor transport | |
| | | | | | out of membrane | |
| | | | | | raft /// negative | |
| | | | | | regulation of | |
| | | | | | transforming | |
| | | | | | growth factor- | |
| | | | | | beta3 production | |
| | | | | | /// positive | |
| | | | | | regulation of | |
| | | | | | activated T cell | |
| | | | | | proliferation /// | |
| | | | | | regulation of | |
| | | | | | phosphorylation | |
| | | | | | /// cholesterol | |
| | | | | | homeostasis /// | |
| | | | | | cholesterol | |
| | | | | | homeostasis /// | |
| | | | | | positive | |
| | | | | | regulation of | |
| | | | | | MAP kinase | |
| | | | | | activity /// | |
| | | | | | regulation of | |
| | | | | | MAPKKK | |
| | | | | | cascade /// | |
| | | | | | response to | |
| | | | | | estrogen stimulus | |
| | | | | | /// respiratory | |
| | | | | | burst /// synaptic | |
| | | | | | vesicle | |
| | | | | | endocytosis | |
| 0.713493 | 211256_x_at | 11120 | U90142 | BTN2A1 | lipid metabolic | — |
| | | | | | process | |
| 0.64966 | 211401_s_at | 2263 | AB030078 | FGFR2 | protein amino | nucleotide binding /// protein |
| | | | | | acid | kinase activity /// protein |
| | | | | | phosphorylation | tyrosine kinase activity /// |
| | | | | | /// protein amino | protein tyrosine kinase activity |
| | | | | | acid | /// transmembrane receptor |
| | | | | | phosphorylation | protein tyrosine kinase activity |
| | | | | | /// cell growth | /// receptor activity /// |
| | | | | | | fibroblast growth factor |
| | | | | | | receptor activity /// protein |
| | | | | | | binding /// ATP binding /// |
| | | | | | | heparin binding /// kinase |
| | | | | | | activity /// transferase activity |
| 2.989883 | 212325_at | 22998 | AK027231 | LIMCH1 | actomyosin | actin binding /// zinc ion |
| | | | | | structure | binding /// metal ion binding |
| | | | | | organization and | |
| | | | | | biogenesis | |
| 0.804735 | 212327_at | 22998 | AK026815 | LIMCH1 | actomyosin | actin binding /// zinc ion |
| | | | | | structure | binding /// metal ion binding |
| | | | | | organization and | |
| | | | | | biogenesis | |
| 0.510313 | 212328_at | 22998 | AB029025 | LIMCH1 | actomyosin | actin binding /// zinc ion |
| | | | | | structure | binding /// metal ion binding |
| | | | | | organization and | |
| | | | | | biogenesis | |
| 1.258768 | 212375_at | 57634 | AL563727 | EP400 | chromatin | nucleotide binding /// nucleic |
| | | | | | modification | acid binding /// DNA binding |
| | | | | | | /// helicase activity /// ATP |
| | | | | | | binding /// hydrolase activity |
| −0.534864 | 212792_at | 23333 | AB020684 | DPY19L1 | — | — |
| −0.703959 | 213795_s_at | 5786 | AL121905 | PTPRA | protein amino | phosphoprotein phosphatase |
| | | | | | acid | activity /// protein tyrosine |
| | | | | | phosphorylation | phosphatase activity /// protein |
| | | | | | /// protein amino | tyrosine phosphatase activity |
| | | | | | acid | /// receptor activity /// |
| | | | | | dephosphorylation | transmembrane receptor |
| | | | | | /// transport /// | protein tyrosine phosphatase |
| | | | | | intracellular | activity /// hydrolase activity |
| | | | | | protein transport | /// phosphoric monoester |
| | | | | | /// protein | hydrolase activity |
| | | | | | transport /// | |
| | | | | | dephosphorylation | |
| −0.600266 | 213929_at | — | AL050204 | — | — | — |
| 1.223141 | 214734_at | 23086 | AB014524 | EXPH5 | intracellular | protein binding /// Rab |
| | | | | | protein transport | GTPase binding |
| 0.439197 | 215620_at | 6239 | AU147182 | RREB1 | transcription /// | nucleic acid binding /// DNA |
| | | | | | regulation of | binding /// zinc ion binding /// |
| | | | | | transcription, | transcription activator activity |
| | | | | | DNA-dependent | /// metal ion binding |
| | | | | | /// regulation of | |
| | | | | | transcription, | |
| | | | | | DNA-dependent | |
| | | | | | /// transcription | |
| | | | | | from RNA | |
| | | | | | polymerase II | |
| | | | | | promoter /// Ras | |
| | | | | | protein signal | |
| | | | | | transduction /// | |
| | | | | | multicellular | |
| | | | | | organismal | |
| | | | | | development | |
| 1.320701 | 216379_x_at | 934 | AK000168 | CD24 | response to | signal transducer activity /// |
| | | | | | hypoxia /// cell | protein binding /// protein |
| | | | | | activation /// | binding /// protein kinase |
| | | | | | regulation of | binding /// carbohydrate |
| | | | | | cytokine and | binding /// protein tyrosine |
| | | | | | chemokine | kinase activator activity |
| | | | | | mediated | |
| | | | | | signaling pathway | |
| | | | | | /// regulation of | |
| | | | | | cytokine and | |
| | | | | | chemokine | |
| | | | | | mediated | |
| | | | | | signaling pathway | |
| | | | | | /// response to | |
| | | | | | molecule of | |
| | | | | | bacterial origin /// | |
| | | | | | response to | |
| | | | | | molecule of | |
| | | | | | bacterial origin /// | |
| | | | | | immune response- | |
| | | | | | regulating cell | |
| | | | | | surface receptor | |
| | | | | | signaling pathway | |
| | | | | | /// elevation of | |
| | | | | | cytosolic calcium | |
| | | | | | ion concentration | |
| | | | | | /// neuromuscular | |
| | | | | | synaptic | |
| | | | | | transmission /// | |
| | | | | | induction of | |
| | | | | | apoptosis by | |
| | | | | | intracellular | |
| | | | | | signals /// Wnt | |
| | | | | | receptor signaling | |
| | | | | | pathway /// cell- | |
| | | | | | cell adhesion /// | |
| | | | | | cell migration /// | |
| | | | | | cell migration /// | |
| | | | | | regulation of | |
| | | | | | epithelial cell | |
| | | | | | differentiation /// | |
| | | | | | T cell | |
| | | | | | costimulation /// B | |
| | | | | | cell receptor | |
| | | | | | transport into | |
| | | | | | membrane raft /// | |
| | | | | | chemokine | |
| | | | | | receptor transport | |
| | | | | | out of membrane | |
| | | | | | raft /// negative | |
| | | | | | regulation of | |
| | | | | | transforming | |
| | | | | | growth factor- | |
| | | | | | beta3 production | |
| | | | | | /// positive | |
| | | | | | regulation of | |
| | | | | | activated T cell | |
| | | | | | proliferation /// | |
| | | | | | regulation of | |
| | | | | | phosphorylation | |
| | | | | | /// cholesterol | |
| | | | | | homeostasis /// | |
| | | | | | cholesterol | |
| | | | | | homeostasis /// | |
| | | | | | positive | |
| | | | | | regulation of | |
| | | | | | MAP kinase | |
| | | | | | activity /// | |
| | | | | | regulation of | |
| | | | | | MAPKKK | |
| | | | | | cascade /// | |
| | | | | | response to | |
| | | | | | estrogen stimulus | |
| | | | | | /// respiratory | |
| | | | | | burst /// synaptic | |
| | | | | | vesicle | |
| | | | | | endocytosis | |
| 3.037363 | 216484_x_at | — | L24521 | — | — | — |
| −0.365784 | 218692_at | 55638 | NM_017786 | GOLSYN | — | — |
| 2.742531 | 219100_at | 79991 | NM_024928 | OBFC1 | — | nucleic acid binding |
| 3.519806 | 220144_s_at | 63926 | NM_022096 | ANKRD5 | — | calcium ion binding |
| 1.235322 | 221750_at | 3157 | BG035985 | HMGCS1 | lipid metabolic | catalytic activity /// |
| | | | | | process /// steroid | hydroxymethylglutaryl-CoA |
| | | | | | biosynthetic | synthase activity /// |
| | | | | | process /// | hydroxymethylglutaryl-CoA |
| | | | | | cholesterol | synthase activity /// transferase |
| | | | | | biosynthetic | activity |
| | | | | | process /// | |
| | | | | | metabolic process | |
| | | | | | /// isoprenoid | |
| | | | | | biosynthetic | |
| | | | | | process /// lipid | |
| | | | | | biosynthetic | |
| | | | | | process /// sterol | |
| | | | | | biosynthetic | |
| | | | | | process | |
| 0.442125 | 222278_at | — | AW969655 | — | — | — |

TABLE 2
Pemetrexed Responsivity Predictor Set

| | | Entrez | Representative | Gene | Go biological | Go molecular function |
|---|---|---|---|---|---|---|
| Weight | Probe Set ID | Gene ID | Public ID | Symbol | process term | term |
| 1.796095 | 200677_at | 754 | NM_004339 | PTTG1IP | protein import into | — |
| | | | | | nucleus /// | |
| | | | | | multicellular | |
| | | | | | organismal | |
| | | | | | development | |
| −0.661121 | 200705_s_at | 1933 | NM_001959 | EEF1B2 | translation /// | translation elongation factor |
| | | | | | translational | activity /// translation |
| | | | | | elongation /// | elongation factor activity /// |
| | | | | | translational | protein binding |
| | | | | | elongation | |
| −0.907053 | 200755_s_at | 813 | BF939365 | CALU | — | calcium ion binding /// |
| | | | | | | calcium ion binding /// |
| | | | | | | calcium ion binding |
| 1.410245 | 200757_s_at | 813 | NM_001219 | CALU | — | calcium ion binding /// |
| | | | | | | calcium ion binding /// |
| | | | | | | calcium ion binding |
| 3.03281 | 200782_at | 308 | NM_001154 | ANXA5 | anti-apoptosis /// | phospholipase inhibitor |
| | | | | | signal transduction | activity /// calcium ion |
| | | | | | /// blood coagulation | binding /// protein binding |
| | | | | | /// negative | /// phospholipid binding /// |
| | | | | | regulation of | calcium-dependent |
| | | | | | coagulation | phospholipid binding |
| 2.730474 | 200787_s_at | 8682 | BC002426 | PEA15 | transport /// | sugar:hydrogen symporter |
| | | | | | transport /// | activity /// protein binding |
| | | | | | apoptosis /// anti- | /// protein binding |
| | | | | | apoptosis /// | |
| | | | | | carbohydrate | |
| | | | | | transport /// | |
| | | | | | regulation of | |
| | | | | | apoptosis /// | |
| | | | | | regulation of | |
| | | | | | apoptosis /// | |
| | | | | | negative regulation | |
| | | | | | of glucose import | |
| 1.114737 | 200788_s_at | 8682 | NM_003768 | PEA15 | transport /// | sugar:hydrogen symporter |
| | | | | | transport /// | activity /// protein binding |
| | | | | | apoptosis /// anti- | /// protein binding |
| | | | | | apoptosis /// | |
| | | | | | carbohydrate | |
| | | | | | transport /// | |
| | | | | | regulation of | |
| | | | | | apoptosis /// | |
| | | | | | regulation of | |
| | | | | | apoptosis /// | |
| | | | | | negative regulation | |
| | | | | | of glucose import | |
| −0.413517 | 200859_x_at | 2316 | NM_001456 | FLNA | cell motility /// cell | actin binding /// signal |
| | | | | | surface receptor | transducer activity /// signal |
| | | | | | linked signal | transducer activity /// |
| | | | | | transduction /// | protein binding /// |
| | | | | | nervous system | transcription factor binding |
| | | | | | development /// | /// actin filament binding |
| | | | | | actin cytoskeleton | |
| | | | | | organization and | |
| | | | | | biogenesis /// | |
| | | | | | positive regulation | |
| | | | | | of transcription | |
| | | | | | factor import into | |
| | | | | | nucleus /// positive | |
| | | | | | regulation of I- | |
| | | | | | kappaB kinase/NF- | |
| | | | | | kappaB cascade /// | |
| | | | | | positive regulation | |
| | | | | | of I-kappaB | |
| | | | | | kinase/NF-kappaB | |
| | | | | | cascade /// negative | |
| | | | | | regulation of | |
| | | | | | transcription factor | |
| | | | | | activity | |
| 1.931894 | 200983_x_at | 966 | BF983379 | CD59 | defense response /// | protein binding |
| | | | | | immune response /// | |
| | | | | | cell surface receptor | |
| | | | | | linked signal | |
| | | | | | transduction /// | |
| | | | | | blood coagulation | |
| 1.803006 | 200984_s_at | 966 | X16447 | CD59 | defense response /// | protein binding |
| | | | | | immune response /// | |
| | | | | | cell surface receptor | |
| | | | | | linked signal | |
| | | | | | transduction /// | |
| | | | | | blood coagulation | |
| −0.523167 | 200985_s_at | 966 | NM_000611 | CD59 | defense response /// | protein binding |
| | | | | | immune response /// | |
| | | | | | cell surface receptor | |
| | | | | | linked signal | |
| | | | | | transduction /// | |
| | | | | | blood coagulation | |
| 1.449346 | 201043_s_at | 100128146 | NM_006305 | ANP32A /// | transcription /// | protein binding /// protein |
| | | /// | | LOC100128146 | regulation of | binding |
| | | 8125 | | | transcription, DNA- | |
| | | | | | dependent /// | |
| | | | | | nucleocytoplasmic | |
| | | | | | transport /// | |
| | | | | | nucleocytoplasmic | |
| | | | | | transport /// | |
| | | | | | intracellular | |
| | | | | | signaling cascade | |
| −0.477653 | 201376_s_at | 3185 | AI591354 | HNRNPF | RNA processing /// | nucleotide binding /// |
| | | | | | mRNA processing | nucleic acid binding /// |
| | | | | | /// RNA splicing /// | RNA binding /// RNA |
| | | | | | regulation of RNA | binding /// protein binding |
| | | | | | splicing | |
| 1.702464 | 201445_at | 1266 | NM_001839 | CNN3 | smooth muscle | actin binding /// actin |
| | | | | | contraction /// | binding /// calmodulin |
| | | | | | muscle development | binding /// calmodulin |
| | | | | | /// actomyosin | binding /// tropomyosin |
| | | | | | structure | binding /// troponin C |
| | | | | | organization and | binding |
| | | | | | biogenesis | |
| 1.870895 | 201616_s_at | 800 | AL577531 | CALD1 | cell motility /// | actin binding /// actin |
| | | | | | muscle contraction | binding /// calmodulin |
| | | | | | | binding /// calmodulin |
| | | | | | | binding /// tropomyosin |
| | | | | | | binding /// myosin binding |
| 0.759276 | 201670_s_at | 4082 | M68956 | MARCKS | cell motility | actin binding /// calmodulin |
| | | | | | | binding /// calmodulin |
| | | | | | | binding /// actin filament |
| | | | | | | binding |
| 1.516249 | 201860_s_at | 5327 | NM_000930 | PLAT | protein modification | catalytic activity /// serine- |
| | | | | | process /// | type endopeptidase activity |
| | | | | | proteolysis /// | /// peptidase activity /// |
| | | | | | proteolysis /// blood | plasminogen activator |
| | | | | | coagulation | activity /// hydrolase |
| | | | | | | activity |
| −0.53828 | 202351_at | 3685 | AI093579 | ITGAV | blood vessel | receptor activity /// calcium |
| | | | | | development /// cell | ion binding /// protein |
| | | | | | adhesion /// cell | binding /// protein binding |
| | | | | | adhesion /// cell- | |
| | | | | | matrix adhesion /// | |
| | | | | | integrin-mediated | |
| | | | | | signaling pathway | |
| | | | | | /// integrin-mediated | |
| | | | | | signaling pathway | |
| −0.884884 | 202392_s_at | 23761 | NM_014338 | PISD | phospholipid | phosphatidylserine |
| | | | | | biosynthetic process | decarboxylase activity /// |
| | | | | | | lyase activity /// carboxy- |
| | | | | | | lyase activity |
| 0.548457 | 202445_s_at | 4853 | NM_024408 | NOTCH2 | cell fate | ligand-regulated |
| | | | | | determination /// | transcription factor activity |
| | | | | | transcription /// | /// receptor activity /// |
| | | | | | regulation of | receptor activity /// calcium |
| | | | | | transcription, DNA- | ion binding /// protein |
| | | | | | dependent /// | binding /// protein |
| | | | | | regulation of | heterodimerization activity |
| | | | | | transcription, DNA- | |
| | | | | | dependent /// anti- | |
| | | | | | apoptosis /// | |
| | | | | | induction of | |
| | | | | | apoptosis /// cell | |
| | | | | | cycle arrest /// | |
| | | | | | Notch signaling | |
| | | | | | pathway /// | |
| | | | | | multicellular | |
| | | | | | organismal | |
| | | | | | development /// | |
| | | | | | multicellular | |
| | | | | | organismal | |
| | | | | | development /// | |
| | | | | | nervous system | |
| | | | | | development /// | |
| | | | | | negative regulation | |
| | | | | | of cell proliferation | |
| | | | | | /// organ | |
| | | | | | morphogenesis /// | |
| | | | | | cell growth /// stem | |
| | | | | | cell maintenance /// | |
| | | | | | hemopoiesis /// cell | |
| | | | | | differentiation /// | |
| | | | | | positive regulation | |
| | | | | | of Ras protein | |
| | | | | | signal transduction | |
| | | | | | /// regulation of | |
| | | | | | developmental | |
| | | | | | process | |
| −0.355835 | 202556_s_at | 10445 | NM_006337 | MCRS1 | protein modification | protein binding |
| | | | | | process | |
| −0.313779 | 202572_s_at | 22839 | NM_014902 | DLGAP4 | cell-cell signaling | — |
| −0.468648 | 202716_at | 5770 | NM_002827 | PTPN1 | protein amino acid | phosphoprotein |
| | | | | | dephosphorylation | phosphatase activity /// |
| | | | | | /// signal | protein tyrosine |
| | | | | | transduction /// | phosphatase activity /// |
| | | | | | insulin receptor | protein tyrosine |
| | | | | | signaling pathway | phosphatase activity /// |
| | | | | | /// | receptor activity /// protein |
| | | | | | dephosphorylation | binding /// protein binding |
| | | | | | | /// hydrolase activity /// |
| | | | | | | phosphoric monoester |
| | | | | | | hydrolase activity |
| 0.63583 | 202773_s_at | 6433 | AI023864 | SFRS8 | transcription /// | RNA binding /// protein |
| | | | | | regulation of | binding |
| | | | | | transcription, DNA- | |
| | | | | | dependent /// | |
| | | | | | mRNA splice site | |
| | | | | | selection /// RNA | |
| | | | | | processing /// | |
| | | | | | mRNA processing | |
| | | | | | /// mRNA | |
| | | | | | processing /// RNA | |
| | | | | | splicing | |
| −0.274323 | 203046_s_at | 8914 | NM_003920 | TIMELESS | morphogenesis of | protein binding /// protein |
| | | | | | an epithelium /// | binding /// protein binding |
| | | | | | transcription /// | /// protein |
| | | | | | regulation of | heterodimerization activity |
| | | | | | transcription, DNA- | |
| | | | | | dependent /// | |
| | | | | | response to DNA | |
| | | | | | damage stimulus /// | |
| | | | | | cell cycle /// mitosis | |
| | | | | | /// multicellular | |
| | | | | | organismal | |
| | | | | | development /// | |
| | | | | | circadian rhythm /// | |
| | | | | | circadian rhythm /// | |
| | | | | | detection of abiotic | |
| | | | | | stimulus /// response | |
| | | | | | to abiotic stimulus | |
| | | | | | /// negative | |
| | | | | | regulation of | |
| | | | | | transcription /// | |
| | | | | | regulation of S | |
| | | | | | phase /// regulation | |
| | | | | | of cell proliferation | |
| | | | | | /// rhythmic process | |
| | | | | | /// cell division | |
| −2.465089 | 203091_at | 8880 | NM_003902 | FUBP1 | transcription /// | DNA binding /// single- |
| | | | | | regulation of | stranded DNA binding /// |
| | | | | | transcription, DNA- | transcription factor activity |
| | | | | | dependent /// | /// RNA binding /// protein |
| | | | | | transcription from | binding |
| | | | | | RNA polymerase II | |
| | | | | | promoter | |
| −0.459075 | 203167_at | 7077 | NM_003255 | TIMP2 | negative regulation | enzyme inhibitor activity /// |
| | | | | | of cell proliferation | integrin binding /// protein |
| | | | | | /// regulation of | binding /// protein binding |
| | | | | | cAMP metabolic | /// enzyme activator activity |
| | | | | | process /// | /// metalloendopeptidase |
| | | | | | regulation of | inhibitor activity /// |
| | | | | | MAPKKK cascade | metalloendopeptidase |
| | | | | | /// regulation of | inhibitor activity |
| | | | | | neuron | |
| | | | | | differentiation | |
| −0.747378 | 203234_at | 7378 | NM_003364 | UPP1 | nucleobase, | catalytic activity /// uridine |
| | | | | | nucleoside, | phosphorylase activity /// |
| | | | | | nucleotide and | uridine phosphorylase |
| | | | | | nucleic acid | activity /// transferase |
| | | | | | metabolic process /// | activity /// transferase |
| | | | | | nucleoside | activity, transferring |
| | | | | | metabolic process /// | glycosyl groups |
| | | | | | nucleotide catabolic | |
| | | | | | process | |
| 0.973201 | 203317_at | 23550 | NM_012455 | PSD4 | regulation of ARF | guanyl-nucleotide exchange |
| | | | | | protein signal | factor activity /// ARF |
| | | | | | transduction | guanyl-nucleotide exchange |
| | | | | | | factor activity |
| −0.294234 | 203322_at | 22850 | AU145934 | ADNP2 | transcription /// | nucleic acid binding /// |
| | | | | | regulation of | DNA binding /// |
| | | | | | transcription, DNA- | transcription factor activity |
| | | | | | dependent | /// zinc ion binding /// |
| | | | | | | sequence-specific DNA |
| | | | | | | binding /// metal ion |
| | | | | | | binding |
| −0.369116 | 203383_s_at | 2800 | BG111661 | GOLGA1 | — | — |
| −0.835708 | 203670_at | 26140 | NM_015644 | TTLL3 | protein modification | actin binding /// tubulin- |
| | | | | | process /// actin | tyrosine ligase activity /// |
| | | | | | filament | structural constituent of |
| | | | | | polymerization /// | cytoskeleton /// protein |
| | | | | | actin nucleation | binding /// protein binding, |
| | | | | | | bridging /// actin filament |
| | | | | | | binding |
| −0.793991 | 203737_s_at | 23082 | NM_015062 | PPRC1 | transcription /// | nucleotide binding /// |
| | | | | | regulation of | nucleic acid binding /// |
| | | | | | transcription, DNA- | RNA binding |
| | | | | | dependent | |
| −0.981388 | 203785_s_at | 55794 | NM_018380 | DDX28 | — | nucleotide binding /// |
| | | | | | | nucleic acid binding /// |
| | | | | | | RNA binding /// helicase |
| | | | | | | activity /// ATP binding /// |
| | | | | | | ATP-dependent helicase |
| | | | | | | activity /// hydrolase |
| | | | | | | activity |
| 2.117176 | 203826_s_at | 9600 | NM_004910 | PITPNM1 | lipid metabolic | calcium ion binding /// |
| | | | | | process /// transport | phosphatidylinositol |
| | | | | | /// brain | transporter activity /// metal |
| | | | | | development /// | ion binding |
| | | | | | phototransduction | |
| | | | | | /// protein transport | |
| −0.556107 | 203832_at | 6636 | NM_003095 | SNRPF | mRNA processing | RNA binding /// RNA |
| | | | | | /// RNA splicing /// | binding /// protein binding |
| | | | | | RNA splicing /// | |
| | | | | | mRNA metabolic | |
| | | | | | process | |
| 0.747164 | 204030_s_at | 29970 | NM_014575 | SCHIP1 | — | protein binding /// identical |
| | | | | | | protein binding |
| −0.305906 | 205053_at | 5557 | NM_000946 | PRIM1 | DNA replication /// | DNA primase activity /// |
| | | | | | DNA replication, | DNA primase activity /// |
| | | | | | synthesis of RNA | DNA-directed RNA |
| | | | | | primer /// DNA | polymerase activity /// |
| | | | | | replication, | protein binding /// zinc ion |
| | | | | | synthesis of RNA | binding /// transferase |
| | | | | | primer /// | activity /// |
| | | | | | transcription | nucleotidyltransferase |
| | | | | | | activity /// metal ion |
| | | | | | | binding |
| 1.235028 | 205079_s_at | 8777 | NM_003829 | MPDZ | — | protein binding /// protein |
| | | | | | | binding /// protein binding |
| 0.602186 | 205424_at | 9755 | NM_014726 | TBKBP1 | immune response /// | — |
| | | | | | innate immune | |
| | | | | | response | |
| 0.311966 | 205702_at | 10745 | NM_006608 | PHTF1 | transcription /// | DNA binding /// |
| | | | | | regulation of | transcription factor activity |
| | | | | | transcription, DNA- | |
| | | | | | dependent | |
| 2.629866 | 205768_s_at | 11001 | NM_003645 | SLC27A2 | very-long-chain | nucleotide binding /// |
| | | | | | fatty acid metabolic | catalytic activity /// long- |
| | | | | | process /// lipid | chain-fatty-acid-CoA ligase |
| | | | | | metabolic process /// | activity /// long-chain-fatty- |
| | | | | | fatty acid metabolic | acid-CoA ligase activity /// |
| | | | | | process /// metabolic | ligase activity |
| | | | | | process | |
| 1.543372 | 205769_at | 11001 | NM_003645 | SLC27A2 | very-long-chain | nucleotide binding /// |
| | | | | | fatty acid metabolic | catalytic activity /// long- |
| | | | | | process /// lipid | chain-fatty-acid-CoA ligase |
| | | | | | metabolic process /// | activity /// long-chain-fatty- |
| | | | | | fatty acid metabolic | acid-CoA ligase activity /// |
| | | | | | process /// metabolic | ligase activity |
| | | | | | process | |
| 1.358871 | 206499_s_at | 1104 /// | NM_001269 | RCC1 /// | G1/S transition of | chromatin binding /// |
| | | 751867 | | SNHG3- | mitotic cell cycle /// | guanyl-nucleotide exchange |
| | | | | RCC1 | DNA packaging /// | factor activity /// Ran |
| | | | | | cell cycle /// mitotic | guanyl-nucleotide exchange |
| | | | | | spindle organization | factor activity /// protein |
| | | | | | and biogenesis /// | binding /// histone binding |
| | | | | | mitosis /// regulation | |
| | | | | | of mitosis /// | |
| | | | | | regulation of S | |
| | | | | | phase of mitotic cell | |
| | | | | | cycle /// cell | |
| | | | | | division | |
| −1.181678 | 206526_at | 26150 | NM_015653 | RIBC2 | — | — |
| 0.515563 | 206983_at | 1235 | NM_004367 | CCR6 | cell motility /// | rhodopsin-like receptor |
| | | | | | chemotaxis /// | activity /// signal transducer |
| | | | | | immune response /// | activity /// receptor activity |
| | | | | | humoral immune | /// receptor activity /// G- |
| | | | | | response /// cellular | protein coupled receptor |
| | | | | | defense response /// | activity /// angiotensin type |
| | | | | | signal transduction | II receptor activity /// |
| | | | | | /// signal | chemokine receptor activity |
| | | | | | transduction /// G- | /// protein binding /// C-C |
| | | | | | protein coupled | chemokine receptor activity |
| | | | | | receptor protein | |
| | | | | | signaling pathway | |
| | | | | | /// elevation of | |
| | | | | | cytosolic calcium | |
| | | | | | ion concentration | |
| −0.367751 | 207416_s_at | 4775 | NM_004555 | NFATC3 | transcription /// | DNA binding /// |
| | | | | | regulation of | transcription factor activity |
| | | | | | transcription, DNA- | /// transcription coactivator |
| | | | | | dependent /// | activity |
| | | | | | regulation of | |
| | | | | | transcription from | |
| | | | | | RNA polymerase II | |
| | | | | | promoter /// | |
| | | | | | transcription from | |
| | | | | | RNA polymerase II | |
| | | | | | promoter /// | |
| | | | | | inflammatory | |
| | | | | | response /// | |
| | | | | | regulation of | |
| | | | | | transcription | |
| −0.142708 | 208008_at | 26083 | NM_015594 | TBC1D29 | regulation of Rab | Rab GTPase activator |
| | | | | | GTPase activity | activity |
| −0.659505 | 209009_at | 2098 | BC001169 | ESD | release of | carboxylesterase activity /// |
| | | | | | cytochrome c from | carboxylesterase activity /// |
| | | | | | mitochondria /// | death receptor binding /// |
| | | | | | apoptosis /// | protein binding /// protein |
| | | | | | induction of | binding /// hydrolase |
| | | | | | apoptosis via death | activity /// S- |
| | | | | | domain receptors /// | formylglutathione |
| | | | | | apoptotic | hydrolase activity |
| | | | | | mitochondrial | |
| | | | | | changes /// | |
| | | | | | regulation of | |
| | | | | | apoptosis /// positive | |
| | | | | | regulation of | |
| | | | | | apoptosis /// neuron | |
| | | | | | apoptosis | |
| 2.819726 | 209286_at | 10602 | AI754416 | CDC42EP3 | signal transduction | protein binding /// |
| | | | | | /// regulation of cell | cytoskeletal regulatory |
| | | | | | shape | protein binding |
| −0.350809 | 209288_s_at | 10602 | AL136842 | CDC42EP3 | signal transduction | protein binding /// |
| | | | | | /// regulation of cell | cytoskeletal regulatory |
| | | | | | shape | protein binding |
| 1.311676 | 209632_at | 5523 | AI760130 | PPP2R3A | protein amino acid | calcium ion binding /// |
| | | | | | dephosphorylation | protein binding /// protein |
| | | | | | | binding /// protein |
| | | | | | | phosphatase type 2A |
| | | | | | | regulator activity |
| −0.130269 | 209633_at | 5523 | AL389975 | PPP2R3A | protein amino acid | calcium ion binding /// |
| | | | | | dephosphorylation | protein binding /// protein |
| | | | | | | binding /// protein |
| | | | | | | phosphatase type 2A |
| | | | | | | regulator activity |
| 0.480937 | 209832_s_at | 81620 | AF321125 | CDT1 | DNA replication | DNA binding /// DNA |
| | | | | | checkpoint /// DNA | binding /// protein binding |
| | | | | | replication | /// protein binding |
| | | | | | checkpoint /// DNA | |
| | | | | | replication /// cell | |
| | | | | | cycle /// regulation | |
| | | | | | of S phase of | |
| | | | | | mitotic cell cycle /// | |
| | | | | | regulation of DNA | |
| | | | | | replication initiation | |
| | | | | | /// regulation of | |
| | | | | | DNA replication | |
| | | | | | initiation | |
| −0.506424 | 210921_at | — | BC002821 | — | — | — |
| 1.733509 | 211612_s_at | 3597 | U62858 | IL13RA1 | cell surface receptor | receptor activity /// |
| | | | | | linked signal | hematopoietin/interferon- |
| | | | | | transduction | class (D200-domain) |
| | | | | | | cytokine receptor activity |
| | | | | | | /// protein binding |
| 1.890649 | 212077_at | 800 | AL583520 | CALD1 | cell motility /// | actin binding /// actin |
| | | | | | muscle contraction | binding /// calmodulin |
| | | | | | | binding /// calmodulin |
| | | | | | | binding /// tropomyosin |
| | | | | | | binding /// myosin binding |
| 0.450398 | 212463_at | 966 | BE379006 | CD59 | defense response /// | protein binding |
| | | | | | immune response /// | |
| | | | | | cell surface receptor | |
| | | | | | linked signal | |
| | | | | | transduction /// | |
| | | | | | blood coagulation | |
| 2.685816 | 212607_at | 10000 | N32526 | AKT3 | protein amino acid | nucleotide binding /// |
| | | | | | phosphorylation /// | protein kinase activity /// |
| | | | | | protein amino acid | protein kinase activity /// |
| | | | | | phosphorylation /// | protein serine/threonine |
| | | | | | signal transduction | kinase activity /// protein |
| | | | | | | binding /// ATP binding /// |
| | | | | | | kinase activity /// |
| | | | | | | transferase activity |
| 0.317675 | 212626_x_at | 3183 | AA664258 | HNRNPC | nuclear mRNA | nucleotide binding /// |
| | | | | | splicing, via | nucleic acid binding /// |
| | | | | | spliceosome /// | RNA binding /// RNA |
| | | | | | mRNA processing | binding /// protein binding |
| | | | | | /// RNA splicing /// | /// identical protein binding |
| | | | | | RNA splicing | |
| −0.9901 | 212724_at | 390 | BG054844 | RND3 | cell adhesion /// | nucleotide binding /// |
| | | | | | small GTPase | GTPase activity /// GTP |
| | | | | | mediated signal | binding /// GTP binding |
| | | | | | transduction /// actin | |
| | | | | | cytoskeleton | |
| | | | | | organization and | |
| | | | | | biogenesis | |
| 0.893335 | 212845_at | 23034 | AB028976 | SAMD4A | positive regulation | translation repressor |
| | | | | | of translation | activity |
| −0.435645 | 212923_s_at | 221749 | AK024828 | C6orf145 | cell communication | protein binding /// |
| | | | | | | phosphoinositide binding |
| 2.326662 | 212962_at | 85360 | AK023573 | SYDE1 | signal transduction | GTPase activator activity /// |
| | | | | | /// activation of Rho | Rho GTPase activator |
| | | | | | GTPase activity | activity |
| −0.304324 | 213139_at | 6591 | AI572079 | SNAI2 | negative regulation | nucleic acid binding /// |
| | | | | | of transcription | DNA binding /// zinc ion |
| | | | | | from RNA | binding /// metal ion |
| | | | | | polymerase II | binding |
| | | | | | promoter /// | |
| | | | | | transcription /// | |
| | | | | | regulation of | |
| | | | | | transcription, DNA- | |
| | | | | | dependent /// | |
| | | | | | multicellular | |
| | | | | | organismal | |
| | | | | | development /// | |
| | | | | | ectoderm and | |
| | | | | | mesoderm | |
| | | | | | interaction /// | |
| | | | | | sensory perception | |
| | | | | | of sound /// | |
| | | | | | response to | |
| | | | | | radiation | |
| 3.220233 | 213196_at | 23361 | AI924293 | ZNF629 | transcription /// | nucleic acid binding /// |
| | | | | | regulation of | DNA binding /// zinc ion |
| | | | | | transcription, DNA- | binding /// metal ion |
| | | | | | dependent | binding |
| −0.250186 | 213202_at | 9739 | N30342 | SETD1A | transcription /// | nucleotide binding /// |
| | | | | | regulation of | nucleic acid binding /// |
| | | | | | transcription, DNA- | RNA binding /// protein |
| | | | | | dependent /// | binding /// |
| | | | | | chromatin | methyltransferase activity |
| | | | | | modification | /// transferase activity /// |
| | | | | | | histone-lysine N- |
| | | | | | | methyltransferase activity |
| −0.545159 | 213306_at | 8777 | AA917899 | MPDZ | — | protein binding /// protein |
| | | | | | | binding /// protein binding |
| −0.619417 | 213731_s_at | 6929 | AI871234 | TCF3 | B cell lineage | DNA binding /// DNA |
| | | | | | commitment /// | binding /// DNA binding /// |
| | | | | | transcription /// | transcription factor activity |
| | | | | | regulation of | /// transcription factor |
| | | | | | transcription, DNA- | activity /// transcription |
| | | | | | dependent /// | factor activity /// protein |
| | | | | | regulation of | binding /// transcription |
| | | | | | transcription, DNA- | regulator activity /// protein |
| | | | | | dependent /// B cell | homodimerization activity |
| | | | | | differentiation /// | /// bHLH transcription |
| | | | | | regulation of | factor binding /// protein |
| | | | | | transcription /// | heterodimerization activity |
| | | | | | positive regulation | /// protein |
| | | | | | of transcription, | heterodimerization activity |
| | | | | | DNA-dependent | |
| −0.691512 | 213746_s_at | 2316 | AW051856 | FLNA | cell motility /// cell | actin binding /// signal |
| | | | | | surface receptor | transducer activity /// signal |
| | | | | | linked signal | transducer activity /// |
| | | | | | transduction /// | protein binding /// |
| | | | | | nervous system | transcription factor binding |
| | | | | | development /// | /// actin filament binding |
| | | | | | actin cytoskeleton | |
| | | | | | organization and | |
| | | | | | biogenesis /// | |
| | | | | | positive regulation | |
| | | | | | of transcription | |
| | | | | | factor import into | |
| | | | | | nucleus /// positive | |
| | | | | | regulation of I- | |
| | | | | | kappaB kinase/NF- | |
| | | | | | kappaB cascade /// | |
| | | | | | positive regulation | |
| | | | | | of I-kappaB | |
| | | | | | kinase/NF-kappaB | |
| | | | | | cascade /// negative | |
| | | | | | regulation of | |
| | | | | | transcription factor | |
| | | | | | activity | |
| 0.990222 | 214240_at | 51083 | AL556409 | GAL | smooth muscle | hormone activity /// |
| | | | | | contraction /// | neuropeptide hormone |
| | | | | | response to stress /// | activity |
| | | | | | inflammatory | |
| | | | | | response /// | |
| | | | | | neuropeptide | |
| | | | | | signaling pathway | |
| | | | | | /// nervous system | |
| | | | | | development /// | |
| | | | | | feeding behavior /// | |
| | | | | | negative regulation | |
| | | | | | of cell proliferation | |
| | | | | | /// insulin secretion | |
| | | | | | /// growth hormone | |
| | | | | | secretion /// | |
| | | | | | regulation of | |
| | | | | | glucocorticoid | |
| | | | | | metabolic process /// | |
| | | | | | response to insulin | |
| | | | | | stimulus /// response | |
| | | | | | to drug /// positive | |
| | | | | | regulation of | |
| | | | | | apoptosis /// | |
| | | | | | response to estrogen | |
| | | | | | stimulus /// negative | |
| | | | | | regulation of | |
| | | | | | lymphocyte | |
| | | | | | proliferation | |
| −0.803423 | 214737_x_at | 3183 | AV725195 | HNRNPC | nuclear mRNA | nucleotide binding /// |
| | | | | | splicing, via | nucleic acid binding /// |
| | | | | | spliceosome /// | RNA binding /// RNA |
| | | | | | mRNA processing | binding /// protein binding |
| | | | | | /// RNA splicing /// | /// identical protein binding |
| | | | | | RNA splicing | |
| −0.290602 | 214752_x_at | 2316 | AI625550 | FLNA | cell motility /// cell | actin binding /// signal |
| | | | | | surface receptor | transducer activity /// signal |
| | | | | | linked signal | transducer activity /// |
| | | | | | transduction /// | protein binding /// |
| | | | | | nervous system | transcription factor binding |
| | | | | | development /// | /// actin filament binding |
| | | | | | actin cytoskeleton | |
| | | | | | organization and | |
| | | | | | biogenesis /// | |
| | | | | | positive regulation | |
| | | | | | of transcription | |
| | | | | | factor import into | |
| | | | | | nucleus /// positive | |
| | | | | | regulation of I- | |
| | | | | | kappaB kinase/NF- | |
| | | | | | kappaB cascade /// | |
| | | | | | positive regulation | |
| | | | | | of I-kappaB | |
| | | | | | kinase/NF-kappaB | |
| | | | | | cascade /// negative | |
| | | | | | regulation of | |
| | | | | | transcription factor | |
| | | | | | activity | |
| −0.687237 | 214880_x_at | 800 | D90453 | CALD1 | cell motility /// | actin binding /// actin |
| | | | | | muscle contraction | binding /// calmodulin |
| | | | | | | binding /// calmodulin |
| | | | | | | binding /// tropomyosin |
| | | | | | | binding /// myosin binding |
| −0.265948 | 215502_at | — | R37655 | — | — | — |
| 1.023266 | 215741_x_at | 26993 | AB015332 | AKAP8L | — | DNA binding /// protein |
| | | | | | | binding /// zinc ion binding |
| | | | | | | /// DEAD/H-box RNA |
| | | | | | | helicase binding /// metal |
| | | | | | | ion binding |
| 2.470912 | 215747_s_at | 1104 /// | X06130 | RCC1 /// | G1/S transition of | chromatin binding /// |
| | | 751867 | | SNHG3- | mitotic cell cycle /// | guanyl-nucleotide exchange |
| | | | | RCC1 | DNA packaging /// | factor activity /// Ran |
| | | | | | cell cycle /// mitotic | guanyl-nucleotide exchange |
| | | | | | spindle organization | factor activity /// protein |
| | | | | | and biogenesis /// | binding /// histone binding |
| | | | | | mitosis /// regulation | |
| | | | | | of mitosis /// | |
| | | | | | regulation of S | |
| | | | | | phase of mitotic cell | |
| | | | | | cycle /// cell | |
| | | | | | division | |
| 1.855822 | 216232_s_at | 10985 | AI697055 | GCN1L1 | regulation of | binding /// protein binding |
| | | | | | translation | /// protein binding /// |
| | | | | | | translation factor activity, |
| | | | | | | nucleic acid binding |
| −0.507409 | 216272_x_at | 85360 | AF209931 | SYDE1 | signal transduction | GTPase activator activity /// |
| | | | | | /// activation of Rho | Rho GTPase activator |
| | | | | | GTPase activity | activity |
| −0.506843 | 216889_s_at | 3172 | Z49825 | HNF4A | transcription /// | DNA binding /// DNA |
| | | | | | regulation of | binding /// transcription |
| | | | | | transcription, DNA- | factor activity /// |
| | | | | | dependent /// | transcription factor activity |
| | | | | | regulation of | /// transcription factor |
| | | | | | transcription from | activity /// RNA |
| | | | | | RNA polymerase II | polymerase II transcription |
| | | | | | promoter /// | factor activity /// steroid |
| | | | | | ornithine metabolic | hormone receptor activity |
| | | | | | process /// lipid | /// receptor activity /// |
| | | | | | metabolic process /// | ligand-dependent nuclear |
| | | | | | xenobiotic | receptor activity /// receptor |
| | | | | | metabolic process /// | binding /// steroid binding |
| | | | | | blood coagulation /// | /// fatty acid binding /// |
| | | | | | blood coagulation /// | protein binding /// zinc ion |
| | | | | | negative regulation | binding /// tRNA- |
| | | | | | of cell proliferation | pseudouridine synthase |
| | | | | | /// positive | activity /// protein |
| | | | | | regulation of | homodimerization activity |
| | | | | | specific | /// sequence-specific DNA |
| | | | | | transcription from | binding /// metal ion |
| | | | | | RNA polymerase II | binding |
| | | | | | promoter /// | |
| | | | | | regulation of lipid | |
| | | | | | metabolic process /// | |
| | | | | | negative regulation | |
| | | | | | of cell growth /// | |
| | | | | | positive regulation | |
| | | | | | of transcription | |
| | | | | | from RNA | |
| | | | | | polymerase II | |
| | | | | | promoter /// lipid | |
| | | | | | homeostasis | |
| −0.211925 | 217203_at | 2752 | U08626 | GLUL | regulation of | catalytic activity /// |
| | | | | | neurotransmitter | glutamate-ammonia ligase |
| | | | | | levels /// glutamine | activity /// ligase activity |
| | | | | | biosynthetic process | |
| | | | | | /// nitrogen | |
| | | | | | compound | |
| | | | | | metabolic process | |
| −0.216946 | 217912_at | 64118 | NM_022156 | DUS1L | tRNA processing /// | catalytic activity /// |
| | | | | | metabolic process | oxidoreductase activity /// |
| | | | | | | tRNA dihydrouridine |
| | | | | | | synthase activity /// FAD |
| | | | | | | binding |
| −0.442052 | 218083_at | 80142 | NM_025072 | PTGES2 | prostaglandin | protein binding /// electron |
| | | | | | biosynthetic process | carrier activity /// protein |
| | | | | | /// fatty acid | disulfide oxidoreductase |
| | | | | | biosynthetic process | activity /// isomerase |
| | | | | | /// lipid biosynthetic | activity /// prostaglandin-E |
| | | | | | process /// cell | synthase activity |
| | | | | | redox homeostasis | |
| −0.825794 | 218275_at | 1468 | NM_012140 | SLC25A10 | gluconeogenesis /// | dicarboxylic acid |
| | | | | | transport /// | transmembrane transporter |
| | | | | | dicarboxylic acid | activity /// binding /// |
| | | | | | transport /// | secondary active |
| | | | | | mitochondrial | transmembrane transporter |
| | | | | | transport | activity |
| 2.028436 | 218330_s_at | 89797 | NM_018162 | NAV2 | sodium ion transport | nucleotide binding /// |
| | | | | | /// small GTPase | helicase activity /// voltage- |
| | | | | | mediated signal | gated sodium channel |
| | | | | | transduction /// | activity /// ATP binding /// |
| | | | | | protein transport | GTP binding /// hydrolase |
| | | | | | | activity /// nucleoside- |
| | | | | | | triphosphatase activity |
| 1.22202 | 218524_at | 1877 | NM_004424 | E4F1 | regulation of cell | nucleic acid binding /// |
| | | | | | growth /// | DNA binding /// DNA |
| | | | | | transcription /// | binding /// transcription |
| | | | | | regulation of | factor activity /// |
| | | | | | transcription, DNA- | transcription coactivator |
| | | | | | dependent /// | activity /// transcription |
| | | | | | ubiquitin cycle /// | corepressor activity /// |
| | | | | | cell cycle /// mitosis | protein binding /// zinc ion |
| | | | | | /// cell proliferation | binding /// ligase activity /// |
| | | | | | /// cell division | metal ion binding |
| 1.413713 | 218630_at | 54903 | NM_017777 | MKS1 | — | — |
| 1.526569 | 218656_s_at | 10186 | NM_005780 | LHFP | — | DNA binding |
| −1.202196 | 218670_at | 80324 | NM_025215 | PUS1 | pseudouridine | tRNA binding /// |
| | | | | | synthesis /// tRNA | pseudouridylate synthase |
| | | | | | processing /// tRNA | activity /// tRNA- |
| | | | | | processing | pseudouridine synthase |
| | | | | | | activity /// isomerase |
| | | | | | | activity |
| −0.456833 | 218860_at | 79050 | NM_024078 | NOC4L | — | protein binding /// protein |
| | | | | | | binding |
| −0.652216 | 218921_at | 59307 | NM_021805 | SIGIRR | negative regulation | rhodopsin-like receptor |
| | | | | | of cytokine and | activity /// transmembrane |
| | | | | | chemokine | receptor activity /// protein |
| | | | | | mediated signaling | binding |
| | | | | | pathway /// acute- | |
| | | | | | phase response /// | |
| | | | | | signal transduction | |
| | | | | | /// G-protein | |
| | | | | | coupled receptor | |
| | | | | | protein signaling | |
| | | | | | pathway /// negative | |
| | | | | | regulation of | |
| | | | | | lipopolysaccharide- | |
| | | | | | mediated signaling | |
| | | | | | pathway /// negative | |
| | | | | | regulation of | |
| | | | | | transcription factor | |
| | | | | | activity /// negative | |
| | | | | | regulation of | |
| | | | | | chemokine | |
| | | | | | biosynthetic process | |
| | | | | | /// innate immune | |
| | | | | | response | |
| 1.343706 | 219344_at | 55315 | NM_018344 | SLC29A3 | transport /// | nucleoside transmembrane |
| | | | | | nucleoside transport | transporter activity |
| 0.647329 | 220216_at | 56260 | NM_019607 | C8orf44 | — | — |
| −0.43341 | 221820_s_at | 84148 | AK024102 | MYST1 | chromatin assembly | chromatin binding /// |
| | | | | | or disassembly /// | histone acetyltransferase |
| | | | | | transcription /// | activity /// histone |
| | | | | | regulation of | acetyltransferase activity /// |
| | | | | | transcription, DNA- | protein binding /// |
| | | | | | dependent /// | transcription factor binding |
| | | | | | negative regulation | /// zinc ion binding /// |
| | | | | | of transcription /// | acyltransferase activity /// |
| | | | | | chromatin | acetyltransferase activity /// |
| | | | | | modification /// | transferase activity /// metal |
| | | | | | histone acetylation | ion binding |
| | | | | | /// myeloid cell | |
| | | | | | differentiation /// | |
| | | | | | positive regulation | |
| | | | | | of transcription | |
| −0.490921 | 221895_at | 158747 | AW469184 | MOSPD2 | — | structural molecule activity |
| 3.819572 | 40189_at | 6418 | M93651 | SET | DNA replication /// | protein phosphatase |
| | | | | | nucleosome | inhibitor activity /// protein |
| | | | | | assembly /// | binding /// protein |
| | | | | | nucleosome | phosphatase type 2A |
| | | | | | assembly /// | regulator activity /// histone |
| | | | | | nucleosome | binding |
| | | | | | disassembly /// | |
| | | | | | nucleocytoplasmic | |
| | | | | | transport /// negative | |
| | | | | | regulation of histone | |
| | | | | | acetylation | |
| −0.51547 | 43977_at | 54929 | AI660497 | TMEM161A | — | — |
| −0.622925 | 44702_at | 85360 | R77097 | SYDE1 | signal transduction | GTPase activator activity /// |
| | | | | | /// activation of Rho | Rho GTPase activator |
| | | | | | GTPase activity | activity |
| −0.210901 | 56748_at | 10107 | X90539 | TRIM10 | hemopoiesis | protein binding /// zinc ion |
| | | | | | | binding /// zinc ion binding |
| | | | | | | /// metal ion binding |
| −0.597075 | 64432_at | 51275 | W05463 | C12orf47 | — | — |

TABLE 3
Cisplatin Predictor Cell Lines

| | | Cisplatin |
|---|---|---|
| | | Resistant (Res) |
| Cell Line | Origin | or Sensitive (Sen) |
| 181/85p | pancreas ca [1] | Res |
| 257p | gastric ca [2] | Res |
| A375 | melanoma | Res |
| BT20 | breast ca | Sen |
| C8161 | melanoma | Res |
| CX-2 | colon ca | Res |
| Du145 | prostate ca | Res |
| DV-90 | lung ca | Sen |
| ES-2 | ovarian ca | Res |
| FU-OV-1 | ovarian ca | Sen |
| Hep3B | HCC | Res |
| HRT-18 | colon ca | Res |
| HT-29 | colon ca | Res |
| ME43 | melanoma | Res |
| MeWo | melanoma | Res |
| OAW42 | ovarian ca | Sen |
| OVCAR3 | ovarian ca | Sen |
| R103 | breast ca [*] | Sen |
| R193 | breast ca | Sen |
| SKBR3 | breast ca | Res |
| SKMel19 | melanoma | Res |
| SKOV-3 | ovarian ca | Res |
| SNU182 | HCC | Res |
| SNU423 | HCC | Res |
| SNU449 | HCC | Res |
| SNU475 | HCC | Res |
| SW13 | prostate ca | Res |

Unless otherwise indicated the cell lines are available from ATCC under the cell name shown.
[*], kindly provided by Professor I. Petersen, Institute Pathology, Charité, Berlin.
[1], Chabner, The role of drugs in cancer treatment. In: Chabner, ed. Pharmacologic principles of cancer treatment. Philadelphia: W. B. Saunders, 1982; 3-14.
[2], Dietel, et al. In vitro prediction of cytostatic drug resistance in primary cell cultures of solid malignant tumors. Eur J Cancer 1993; 29A: 416-420.

TABLE 4
Pemetrexed Predictor Cell Lines

| | Resistant (Res) |
|---|---|
| Cell Lines | or Sensitive (Sen) |
| K-562 | Res |
| Molt-4 | Res |
| HL-60 | Res |
| MCF7 | Res |
| HCC-2998 | Res |
| HCT-116 | Res |
| NCI-H460 | Res |
| SNB-19 | Sen |
| HS578T | Sen |
| MDA-MB-231 | Sen |
| MDA-MB-435 | Sen |
| NCI-H226 | Sen |
| M14 | Sen |
| MALME-3M | Sen |
| SK-MEL-2 | Sen |
| SK-MEL-28 | Sen |
| 257P | Res |
| A375 | Res |
| C8161 | Res |
| ES2 | Res |
| me43 | Res |
| SKMel19 | Res |
| SNU182 | Res |
| SNU423 | Res |
| Sw13 | Res |
| BT20 | Sen |
| DV90 | Sen |
| FUOV1 | Sen |
| OAW42 | Sen |
| OVKAR | Sen |
| R103 | Sen |

The cell lines are all available through the National Cancer Institute.

1. A method for predicting responsiveness of a cancer to a platinum-based chemotherapeutic agent comprising:
a. comparing a first gene expression profile of the cancer to a platinum chemotherapy responsivity predictor set of gene expression profiles, the first gene expression profile and the platinum chemotherapy responsivity predictor set each comprising at least 2 genes from Table 1; and
b. using the comparison of step(a) to predict the responsiveness of the cancer to a platinum-based chemotherapeutic agent.

2. The method of claim 1, wherein the first gene expression profile is obtained by analyzing a nucleic acid sample from the cancer.

3. The method of claim 1, wherein the first gene expression profile is obtained by analyzing a sample from a tumor or ascites.

4. The method of claim 1, wherein the first gene expression profile is determined by quantifying nucleic acid levels of genes using a DNA microarray.

5. The method of claim 1, wherein the first gene expression profile and the platinum chemotherapy responsivity predictor set each comprise at least 10 genes from Table 1.

6. The method of claim 1, wherein the cancer is from an individual and wherein step (b) identifies the individual as a complete responder or as an incomplete responder.

7. The method of claim 1, wherein the platinum-based chemotherapeutic agent is cisplatin.

8. The method of claim 1, wherein the cancer is selected from the group consisting of lung, breast, and ovarian cancer.

9. The method of claim 1, wherein step (a) comprises using the platinum chemotherapy responsivity predictor set to define at least one metagene by extracting a single dominant value using singular value decomposition (SVD) and determining the value of the metagene in the cancer.

10. The method of claim 9, wherein step (b) comprises applying one or more statistical models to the values of the metagenes, wherein each model includes a statistical probability of the sensitivity of the cancer to the platinum-based chemotherapeutic agent.

11. The method of claim 10, wherein the statistical model is a binary regression model.

12. The method of claim 10, wherein the statistical model is a tree model, the tree model including one or more nodes, each node representing a metagene, each node including a statistical probability of sensitivity of the cancer to the platinum-based chemotherapeutic agent.

13. A method of predicting responsiveness of a cancer to an antimetabolite chemotherapeutic agent comprising:
a. comparing a first gene expression profile of the cancer to an antimetabolite chemotherapy responsivity predictor set of gene expression profiles, the first gene expression profile and the antimetabolite chemotherapy responsivity predictor set each comprising at least 2 genes from Table 2; and
b. using the comparison of step(a) to predict the responsiveness of the cancer to an antimetabolite chemotherapeutic agent.

14. The method of claim 13, wherein the first gene expression profile is obtained by analyzing a nucleic acid sample from the cancer.

15. The method of claim 13, wherein the first gene expression profile is obtained by analyzing a sample from a tumor or ascites.

16. The method of claim 13, wherein the first gene expression profile is determined by quantifying nucleic acid levels of genes using a DNA microarray.

17. The method of claim 13, wherein the first gene expression profile and the antimetabolite chemotherapy responsivity predictor set each comprise at least 10 genes from Table 2.

18. The method of claim 13, wherein the antimetabolite chemotherapy agent is pemetrexed.

19. The method of claim 13, wherein the cancer is selected from the group consisting of lung, breast and ovarian cancer.

20. The method of claim 13, wherein step (a) comprises using the antimetabolite chemotherapy responsivity predictor set to define at least one metagene by extracting a single dominant value using singular value decomposition (SVD) and determining the value of the metagene in the cancer.

21. The method of claim 20, wherein step (b) comprises applying one or more statistical models to the values of the metagenes, wherein each model includes a statistical probability of the sensitivity of the cancer to the antimetabolite chemotherapeutic agent.

22. The method of claim 21, wherein the statistical model is a binary regression model.

23. The method of claim 21, wherein the statistical model is a tree model, the tree model including one or more nodes, each node representing a metagene, each node including a statistical probability of sensitivity of the cancer to the antimetabolite chemotherapeutic agent.

24. A method of developing a treatment plan for an individual with cancer comprising:
a. using the method of claim 1 to predict responsivity of a cancer to a platinum-based chemotherapeutic agent; and
b. if the cancer is predicted to respond to a platinum-based chemotherapeutic agent, administering an effective amount of a platinum-based chemotherapeutic agent to the individual with the cancer.

25. The method of claim 24, further comprising comparing the first gene expression profile to an alternative chemotherapy responsivity predictor set of gene expression profiles predictive of responsivity to alternative chemotherapeutic agents; predicting responsiveness of the cancer to the alternative chemotherapeutic agents and administering an alternative chemotherapeutic agent to the individual with the cancer, thereby treating the individual with cancer.

26. The method of claim 25, wherein the first gene expression profile and the alternative chemotherapy responsivity predictor set each comprise at least 2 genes from Table 2 and predicts responsivity to antimetabolite chemotherapeutic agents.

27. The method of claim 25, wherein the alternative chemotherapeutic agent is selected from the group comprising docetaxel, paclitaxel, abraxane, topotecan, adriamycin, etoposide, fluorouracil (5-FU), cyclophosphamide, denopterin, edatrexate, methotrexate, nolatrexcd, pemetrexed, piritrexim, pteropterin, raltitrexed, trimetrexate, cladribine, ctofarabine, fludarabine, 6-mercaptopurine, nelarabine, thiamiprine, thioguanine, tiazofurin, ancitabine, azacibdine, 6-azauridine, capecitabine, carmofur, cytarabine, decitabine, doxifluridine, enocitabine, floxuridine, fluorouracil, gemcitabine, tegafur, troxacitabine, pentostatin, hydroxyurea, cytosine arabinoside.

28. The method of claim 24, wherein the platinum-based chemotherapeutic agent is administered before, after or concurrently with the administration of one or more alternative chemotherapeutic agents.

29. A method of developing a treatment plan for an individual with cancer comprising:
a. using the method of claim 13 to predict responsivity of a cancer to an antimetabolite chemotherapeutic agent; and
b. if the cancer is predicted to respond to an antimetabolite chemotherapeutic agent, administering an effective amount of an antimetabolite chemotherapeutic agent to the individual with the cancer.

30. The method of claim 29, wherein the antimetabolite chemotherapy agent is pemetrexed.

31. The method of claim 29, further comprising comparing the first gene expression profile to an alternative chemotherapy responsivity predictor set of gene expression profiles predictive of responsivity to alternative chemotherapeutic agents; predicting responsiveness of the cancer to the alternative chemotherapeutic agents and administering an alternative chemotherapeutic agent to the individual with the cancer, thereby treating the individual with cancer.

32. The method of claim 31, wherein the alternative chemotherapeutic agent is a platinum chemotherapeutic agent.

33. The method of claim 29, wherein the antimetabolite therapy is administered before, after or concurrently with the administration of one or more alternative chemotherapeutic agents.

34.-35. (canceled)

36. A computer readable medium comprising gene expression profiles and corresponding responsivity information for platinum-based chemotherapeutic agents or antimetabolite chemotherapeutic agents comprising at least 5 genes from any of Tables 1 or 2.
